(19) United States
(12) Patent Application Publication
(10) Pub. No.: US 2024/0325528 A1
(43) Pub. Date: Oct. 3, 2024

(54) SUBCUTANEOUS UNIT DOSAGE FORMS

(71) Applicant: argenx BV, Ghent (BE)

(72) Inventors: Antoinetta Jacoba Maria VAN BRAGT, Liempde (NL); Peter Ulrichts, Destelbergen (BE); Erik Hofman, Ghent (BE); Peter Verheesen, Ghent (BE)

(21) Appl. No.: 18/431,585

(22) Filed: Feb. 2, 2024

Related U.S. Application Data
(63) Continuation of application No. PCT/IB2022/000443, filed on Aug. 2, 2022.
(60) Provisional application No. 63/203,856, filed on Aug. 2, 2021.

(51) Int. Cl. A61K 39/395; A61K 9/00; A61K 38/47; C07K 16/28

(52) U.S. Cl. CPC A61K 39/3955; A61K 9/0019; A61K 38/47; C07K 16/283

(57) ABSTRACT

Provided herein are unit dosage forms of a biologic that are determined based on a modeling approach, which matches a pharmacodynamic (PD) value of the SC dose with that of a known reference IV dose, while a pharmacokinetic (PK) value of the SC dose is less than that of the IV dose.

### CROSS REFERENCE TO RELATED APPLICATIONS

This application is a continuation of PCT Application No. PCT/IB2022/000443 filed Aug. 2, 2022, which claims the benefit of U.S. Provisional Application No. 63/203,856, filed Aug. 2, 2021, the contents of these applications are incorporated herein by reference in their entireties.

### SEQUENCE LISTING

The instant application contains a sequence listing which has been submitted electronically in XML ST.26 format and is hereby incorporated by reference in its entirety. Said XML copy, created on Feb. 2, 2024, is named 404373-ARGX-T2101_SL.xml and is 142,255 bytes in size.

### BACKGROUND

Biologics, including antibodies and antibody fragments, are used for treating a wide range of diseases. Intravenous (IV) administration is the primary method of administering many biologics. However, due to the requirements for IV administration, there are issues with patient compliance. Further, due to the chronic nature of many diseases and disorders that are treated with biologics, many patients will require treatment for life, emphasizing the necessity to improve patient compliance. Subcutaneous (SC) administration of biologics is an alternative to IV administration. Compared to IV infusions, SC administration of biologics has several advantages. For example, SC administration reduces the incidence of systemic reactions, lower risk of infections, does not require sometimes-difficult IV access, and is more convenient for patients.

Previously, it was thought that SC administration of biologics, especially those that have a high molecular weight, would lead to reduced bioavailability compared to IV administration, which is common with SC administration of biologics. Generally, the bioavailability of a biologic in human subjects is determined following a single SC dose and a single IV dose. This data is then used in a model to calculate SC dosing, which aims to match the pharmacokinetic (PK) parameters of safe and effective IV doses. Specifically, the goal is to achieve a similar clinical response for the SC dose, compared to the IV dose. However, this approach can lead to high dosage amounts for SC administration, which may not be possible to administer to a patient or could result in increased adverse events in patients.

Accordingly, there is a need in the art for improved methods of determining safe and effective SC doses of biologics.

### SUMMARY

Provided herein are unit dosage forms of a biologic that are determined based on a modeling approach, which matches a pharmacodynamic (PD) value of the SC dose with that of a known reference IV dose, while a pharmacokinetic (PK) value of the SC dose is less than that of the IV dose. The unit dosage forms provided herein show comparable safety and efficacy as compared to a reference IV dose, and are therefore, non-inferior to the IV dose, providing patients with a more convenient alternative method of administration of a biologic.

Previously known methods of determining an SC dose are based on models that aim to match a PK value of an SC dose and a reference IV dose, which leads to unit dosage forms with higher dosage amounts of biologics, as compared to the methods used herein. Accordingly, the unit dosage forms disclosed herein comprise lower dosage amounts of a biologic, which could decrease adverse events in patients and could allow for subcutaneous administration as an alternative for biologics that are generally administered by IV infusion.

Accordingly, provided herein are unit dosage forms for subcutaneous administration of a biologic, wherein the biologic has an RDᵢᵥ, which results in a PKᵢᵥ and a PDᵢᵥ in a subject upon intravenous administration; the unit dosage form comprises an RD_{sc} of the biologic, which results in a PK_{sc} and a PD_{sc} in a subject upon subcutaneous administration; and the ratio PK_{sc}/PKᵢᵥ is less than 0.8 and the ratio PD_{sc}/PDᵢᵥ is from 0.9 to 1.1.

Also provided herein are unit dosage forms for subcutaneous administration of a biologic, wherein the biologic has an RDᵢᵥ, which results in a PKᵢᵥ and a BLᵢᵥ in a subject upon intravenous administration, the unit dosage form comprises an RD_{sc} of the biologic, which results in a PK_{sc} and a BL_{sc} in a subject upon subcutaneous administration; and the ratio PK_{sc}/PKᵢᵥ is less than about 0.8 and the ratio BL_{sc}/BLᵢᵥ is of about 0.9 to about 1.1.

Also provided herein are unit dosage forms for subcutaneous administration of a biologic, wherein the amount subcutaneous dose of the biologic in the unit dosage form was determined by a method comprising the steps of (a) administering a subcutaneous dose of the biologic to a subject, wherein the biologic has an RDᵢᵥ, which results in a PKᵢᵥ and a BLᵢᵥ; (b) determining the BL_{sc}; (c) determining the PK_{sc} of the biologic; and (d) determining a subcutaneous dose that would result in a BL_{sc}/BLᵢᵥ¬ ratio of about 0.9 to about 1.1 and a PK_{sc}/PKᵢᵥ ratio less than about 0.8.

In an embodiment, the BL_{sc} and the BLᵢᵥ are levels of total serum IgG in the subject. In an embodiment, the total serum IgG in the subject is analyzed using a bioanalytical method. In an embodiment, the bioanalytical method is ELISA or automated diagnostic analyzer (IVD).

In an embodiment, the subject is a healthy volunteer or a non-human animal.

In an embodiment, the PDᵢᵥ and the PD_{sc} values are the AUC. In an embodiment, the PK_{sc}/PKᵢᵥ ratio is less than 0.7. In an embodiment, the PK_{sc}/PKᵢᵥ ratio is less than 0.6. In an embodiment, the PK_{sc}/PKᵢᵥ ratio is about 0.8, about 0.7, about 0.6, or about 0.5.

In an embodiment, the PDᵢᵥ and the PD_{sc} values are total serum IgG reduction. In an embodiment, the PD_{sc}/PDᵢᵥ ratio is from 0.9 to 1.1. In an embodiment, the PD_{sc}/PDᵢᵥ ratio is 0.9, 1.0, or 1.1.

In an embodiment, the biologic is selected from the group consisting of antibodies, antibody fragments, anticoagulants, blood factors, bone morphogenetic proteins, enzymes, fusion proteins, growth factors, hormones, interferons, interleukins, and thrombolytics.

In an embodiment, the biologic is an antibody, for example, an anti-FcRn antibody. In an embodiment, the antibody is rozanolixizumab (UCB7665), nipocalimab (M281), orilanolimab (ALXN1830/SYNT001), or batoclimab (IMVT-1401/RVT1401/HBM9161).

In an embodiment, the biologic comprises or consists of a variant Fc region, or FcRn binding fragment thereof, which binds to FcRn with a higher affinity at pH5.5 as compared to a corresponding wild-type Fc region.

In an embodiment, the biologic antagonizes FcRn binding to an antibody Fc region.

In an embodiment, the biologic is efgartigimod.

In an embodiment, the RDᵢᵥ is from 10 mg/kg to 25 mg/kg and the RD_{sc} is from about 1000 mg to about 2000 mg. In an embodiment, the RDᵢᵥ is 10 mg/kg and the RD_{sc} is about 1000 mg. In an embodiment, the RDᵢᵥ is 25 mg/kg and the RD_{sc} is about 2000 mg.

In an embodiment, the unit dosage form further comprises a hyaluronidase enzyme. In an embodiment, the hyaluronidase enzyme comprises an amino acid sequence selected from the group consisting of SEQ ID NO: 5-96. In an embodiment, the hyaluronidase enzyme is rHuPH20.

In an embodiment, the unit dosage form is co-administered with a hyaluronidase enzyme. In an embodiment, the hyaluronidase enzyme is rHuPH20.

In an embodiment, the amount of hyaluronidase enzyme is from about 1000 U/ml to about 3000 U/ml. In an embodiment, the amount of hyaluronidase enzyme is about 1000 U/mL, about 1500 U/mL, about 2000 U/mL, about 2500 U/mL, or about 3000 U/mL. In an embodiment, the amount of hyaluronidase enzyme is 2000 U/mL.

In an embodiment, the unit dosage form is for use in treatment of an autoimmune disease. In an embodiment, the autoimmune disease is selected from the group consisting of allogenic islet graft rejection, alopecia areata, ankylosing spondylitis, antiphospholipid syndrome, autoimmune Addison's disease, Alzheimer's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenic purpura or idiopathic thrombocytopenia purpura or immune mediated thrombocytopenia), autoimmune urticaria, Behcet's disease, bullous pemphigoid (BP), cardiomyopathy, Castleman's syndrome, celiac spruce-dermatitis, chronic fatigue immune disfunction syndrome, chronic inflammatory demyelinating polyneuropathy (CIDP), Churg-Strauss syndrome, cicatricial pemphigoid, CREST syndrome, cold agglutinin disease, Crohn's disease, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic membranous neuropathy, idiopathic pulmonary fibrosis, IgA neuropathy, IgM polyneuropathies, juvenile arthritis, Kawasaki's disease, lichen planus, lichen sclerosus, lupus erythematosus, Meniere's disease, mixed connective tissue disease, mucous membrane pemphigoid, multiple sclerosis, Type 1 diabetes mellitus, multifocal motor neuropathy (MMN), myasthenia gravis (MG), paraneoplastic bullous pemphigoid, pemphigoid gestationis, pemphigus vulgaris (PV), pemphigus foliaceus (PF), pernicious anemia, polyarteritis nodosa, polychrondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Raynaud's phenomenon, Reiter's syndrome, rheumatoid arthritis, sarcoidosis, scleroderma, Sjögren's syndrome, solid organ transplant rejection, stiff-man syndrome, systemic lupus erythematosus, Takayasu's arteritis, toxic epidermal necrolysis (TEN), Stevens-Johnson syndrome (SJS), temporal arteritis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, and Wegner's granulomatosis.

Also provided herein is method of determining a therapeutically effective dose of a biologic for subcutaneous administration, the method comprising: (a) administering a subcutaneous dose of the biologic to a subject, wherein the biologic has an RDᵢᵥ, which results in a PKᵢᵥ and a BLᵢᵥ; (b) determining the BL_{sc} of the biologic; (c) determining the PK_{sc} of the biologic; and (d) determining a subcutaneous dose that would result in a BL_{sc}/BLᵢᵥ ratio of about 0.9 to about 1.1 and a PK_{sc}/PKᵢᵥ ratio less than about 0.8, thereby determining a therapeutically effective dose of the biologic for subcutaneous administration.

In an embodiment, the subject is a healthy volunteer or a non-human animal.

Also provided herein is a method of treating a subject with a subcutaneous dose of a biologic, wherein the subcutaneous dose of the biologic was determined by a method comprising the steps of: (a) administering a subcutaneous dose of the biologic to a subject, wherein the biologic has an RDᵢᵥ, which results in a PKᵢᵥ and a BLᵢᵥ; (b) determining the BL_{sc} of the biologic; (c) determining the PK_{sc} of the biologic; and (d) determining a subcutaneous dose that would result in a BL_{sc}/BLᵢᵥ ratio of about 0.9 to about 1.1 and a PK_{sc}/PKᵢᵥ ratio less than about 0.8.

In an embodiment, the ratio PK_{sc}/PKᵢᵥ is less than 0.7. In an embodiment, the ratio PK_{sc}/PKᵢᵥ is less than 0.6. In an embodiment, the PKᵢᵥ and the PK_{sc} values are the AUC.

In an embodiment, biologic is selected from the group consisting of antibodies, antibody fragments, anticoagulants, blood factors, bone morphogenetic proteins, enzymes, fusion proteins, growth factors, hormones, interferons, interleukins, and thrombolytics.

In an embodiment, the BL_{sc} and the BLᵢᵥ are levels of total serum IgG in the subject. In an embodiment, the total serum IgG in the subject is analyzed using a bioanalytical method. In an embodiment, the bioanalytical method is ELISA or automated diagnostic analyzer (IVD).

In an embodiment, wherein the biologic is an antibody. In an embodiment, the antibody is an anti-FcRn antibody. In an embodiment, the anti-FcRn antibody is rozanolixizumab (UCB7665), nipocalimab (M281), orilanolimab (ALXN1830/SYNT001), or batoclimab (IMVT-1401/RVT1401/HBM9161).

In an embodiment, wherein the biologic comprises or consists of a variant Fc region, or FcRn binding fragment thereof, which binds to FcRn with a higher affinity at pH5.5 as compared to a corresponding wild-type Fc region. In an embodiment, the biologic antagonizes FcRn binding to an antibody Fc region. In an embodiment, wherein the biologic is efgartigimod.

In an embodiment, the RDᵢᵥ is 10 mg/kg. In an embodiment, wherein the RDᵢᵥ is 25 mg/kg.

In an embodiment, the therapeutically effective amount of the biologic is co-administered with a hyaluronidase enzyme. In an embodiment, the therapeutically effective amount of the biologic is administered before or after a hyaluronidase enzyme. In an embodiment, the hyaluronidase enzyme comprises an amino acid sequence selected from the group consisting of SEQ ID NO: 5-96. In an embodiment, the hyaluronidase enzyme is rHuPH20. In an embodiment, the amount of hyaluronidase enzyme is from 1000 U/ml to 3000 U/ml, preferably 2000 U/mL.

Also provided herein is a unit dosage form of a variant Fc region, or FcRn binding fragment thereof, wherein the Fc domains of the Fc region comprise the amino acids Y, T, E, K, F, and Y at EU positions 252, 254, 256, 433, 434, and 436 respectively, for use in treating an autoimmune disease in a human patient.

Also provided herein is a variant Fc region, or FcRn binding fragment thereof, wherein the Fc domains of the Fc region comprise the amino acids Y, T, E, K, F, and Y at EU Kabat positions 252, 254, 256, 433, 434, and 436 respectively, for use in treating myasthenia gravis in a human patient.

In one aspect, the instant disclosure provides a variant Fc region, or FcRn binding fragment thereof, wherein the Fc domains of the Fc region comprise the amino acids Y, T, E, K, F, and Y at EU Kabat positions 252, 254, 256, 433, 434, and 436 respectively, for use in treating myasthenia gravis in a human patient, wherein: the variant Fc region, or FcRn binding fragment thereof, is administered subcutaneously at a weekly dose of between 950 and 1050 mg, independent of the weight of the patient, and a total serum IgG reduction in the patient of at least 60% compared to baseline IgG level is obtained.

In an embodiment, the weekly dose is about 950 mg, about 975 mg, about 1000 mg, about 1025 mg, or about 1050 mg. In an embodiment, the weekly dose is about 1000 mg.

Also provided herein is a variant Fc region, or FcRn binding fragment thereof, wherein the Fc domains of the Fc region comprise the amino acids Y, T, E, K, F, and Y at EU Kabat positions 252, 254, 256, 433, 434, and 436 respectively, for use in treating pemphigus vulgaris in a human patient.

In one aspect, the instant disclosure provides a variant Fc region, or FcRn binding fragment thereof, wherein the Fc domains of the Fc region comprise the amino acids Y, T, E, K, F, and Y at EU Kabat positions 252, 254, 256, 433, 434, and 436 respectively, for use in treating pemphigus vulgaris in a human patient, wherein: the variant Fc region, or FcRn binding fragment thereof, is administered subcutaneously at a weekly dose of between 1950 and 2050 mg, independent of the weight of the patient, and a total serum IgG reduction in the patient of at least 60% compared to baseline IgG level is obtained.

In an embodiment, the weekly dose is about 1950 mg, about 1975 mg, about 2000 mg, about 2025 mg, or about 2050 mg. In an embodiment, the weekly dose is about 2000 mg.

In an embodiment, the treatment comprises at least 2 weekly doses. In an embodiment, the treatment comprises at least 3 weekly doses. In an embodiment, the treatment comprises at least 4 weekly doses. In an embodiment, the treatment comprises at least 5 weekly doses. In an embodiment, the treatment comprises at least 6 weekly doses. In an embodiment, the treatment comprises at least 7 weekly doses. In an embodiment, the treatment comprises at least 8 weekly doses. In an embodiment, the treatment comprises at more than 8 weekly doses.

In an embodiment, the variant Fc region, or FcRn binding fragment thereof, is administered with a hyaluronidase enzyme. In an embodiment, the hyaluronidase enzyme comprises an amino acid sequence selected from the group consisting of SEQ ID NO: 5-96. In an embodiment, the hyaluronidase enzyme is rHuPH20.

In an embodiment, a total serum IgG reduction in the patient of about 60% compared to baseline IgG level is obtained. In an embodiment, a total serum IgG reduction in the patient of about 65%, about 70%, about 75%, or about 80% compared to baseline IgG level is obtained.

In an embodiment, the percentage of total serum IgG reduction in the patient is achieved within 1 month from the first dose. In an embodiment, the percentage of total serum IgG reduction in the patient is achieved within 2 weeks, 3 weeks, 4 weeks, 5 weeks, or 6 weeks from the first dose. In an embodiment, the percentage of total serum IgG reduction in the patient is achieved within 31, 30, 29, 28, 27, 26, or 25 days from the first dose.

In an embodiment, the maximum percentage of total serum IgG reduction in the patient is achieved within 1 month from the first dose. In an embodiment, the maximum percentage of total serum IgG reduction in the patient is achieved within 2 weeks, 3 weeks, 4 weeks, 5 weeks, or 6 weeks from the first dose. In an embodiment, the maximum percentage of total serum IgG reduction in the patient is achieved within 31, 30, 29, 28, 27, 26, or 25 days from the first dose.

In an embodiment, the total serum IgG level in the patient is reduced to 2000 to 4000 μg/mL. In an embodiment, the total serum IgG level in the patient is reduced to 2000 to 3000 μg/mL. In an embodiment, the total serum IgG level in the patient is reduced to 3000 to 4000 μg/mL. In an embodiment, the total serum IgG level in the patient is reduced to 2500 to 3500 μg/mL. In an embodiment, the total serum IgG level in the patient is reduced to 2750 to 3250 μg/mL.

In an embodiment, the total serum IgG in the patient is analyzed using a bioanalytical method. In an embodiment, the total serum IgG in the patient is analyzed using ELISA or automated diagnostic analyzer (IVD).

In an embodiment, at least one of the IgG subtypes is reduced. In an embodiment, IgG1 is reduced. In an embodiment, IgG2 is reduced. In an embodiment, IgG3 is reduced. In an embodiment, IgG4 is reduced.

In an embodiment, the variant Fc region is efgartigimod.

### BRIEF DESCRIPTION OF THE DRAWINGS

FIGS. 1A-1B are graphs showing serum efgartigimod levels in patients from historical data following IV and SC administration of efgartigimod with or without rHuPH20 (FIG. 1A) and following SC co-administration of efgartigimod with rHuPH20 (FIG. 1B).

FIGS. 2A-C are graphs showing total IgG reduction following single SC doses of 750 mg (FIG. 2A), 1250 mg (FIG. 2B), and 1750 mg (FIG. 2C), co-administered with rHuPH20, compared to historical data following an SC dose of 10 mg/kg, without rHuPH20, and an IV dose of 10 mg/kg, without rHuPH20.

FIG. 3 is a graph showing visual predictive checks of efgartigimod concentration in the study described in Example 1. Grey dots are observed data; blue solid line is observed median; red dashed lines are 10^{th} and 90^{th} percentiles of observations; grey area is 80% prediction interval.

FIG. 4 is a graph showing a visual predictive check of efgartigimod concentration on log-scale in a previous study. Grey dots are observed data; blue solid line is observed median; red dashed lines are 10^{th} and 90^{th} percentiles of observations; grey area is 80% prediction interval.

FIG. 5 is a graph showing a comparison of 10 mg/kg SC of efgartigimod without rHuPH20 (blue lines) and with rHuPH20 (red lines). Blue dots are observations from healthy volunteers receiving 10 mg/kg SC efgartigimod without rHuPH20; red dots are observations from healthy volunteers receiving 10 mg/kg SC efgartigimod in combination with rHuPH20; blue lines are population predictions of efgartigimod concentration without rHuPH20; red lines are population predictions of efgartigimod concentration with rHuPH20.

FIG. 6 is a graph showing visual predictive checks of total IgG concentrations in the study described in Example 1, obtained with the PK/PD model in which the parameters were optimized using data from previous studies. Grey dots are observed data; blue solid line is observed median; red dashed lines are 10^{th} and 90^{th} percentiles of observations; grey area is 80% prediction interval.

FIG. 7 is a graph showing visual predictive checks of total IgG reduction in the study described in Example 1, obtained with the PK/PD model in which the parameters were optimized on data from previous studies. Grey dots are observed data; blue solid line is observed median; red dashed lines are 10^{th} and 90^{th} percentiles of observations; grey area is 80% prediction interval.

FIG. 8 is a graph showing visual predictive checks of total IgG concentrations in the study described in Example 1, obtained with the PK/PD model accounting for the effect compartment. Grey dots are observed data; blue solid line is observed median; red dashed lines are 10^{th} and 90^{th} percentiles of observations; grey area is 80% prediction interval.

FIG. 9 is a graph showing visual predictive checks of total IgG reduction in the study described in Example 1, obtained with the PK/PD model accounting for the effect compartment. Grey dots are observed data; blue solid line is observed median; red dashed lines are 10^{th} and 90^{th} percentiles of observations; grey area is 80% prediction interval.

FIG. 10 is a graph showing visual predictive checks of total IgG concentrations in historical data, obtained with the PK/PD model accounting for the effect compartment. Grey dots are observed data; blue solid line is observed median; red dashed lines are 10^{th} and 90^{th} percentiles of observations; grey area is 80% prediction interval.

FIG. 11 is a graph showing visual predictive checks of total IgG reduction in historical data, obtained with the PK/PD model accounting for the effect compartment. Grey dots are observed data; blue solid line is observed median; red dashed lines are 10^{th} and 90^{th} percentiles of observations; grey area is 80% prediction interval.

FIG. 12 is a graph showing the area under the effect curve (AUEC) between day 22 and 29 determined from the total IgG reduction simulations. The solid and dashed horizontal lines are the median and 90% CI of AUEC between day 22 and 29 obtained with the 10 mg/kg IV QW dose of efgartigimod. The points and bars are the median and 90% CI of AUEC between day 22 and 29 obtained with the SC QW doses of efgartigimod.

FIG. 13 is a graph showing the simulated maximum total IgG reduction between day 22 and 29. The solid and dashed horizontal lines are the median and 90% CI of maximum total IgG reduction obtained with the 10 mg/kg IV QW dose of efgartigimod. The points and bars are the median and 90% CI of total IgG reduction obtained with the SC QW doses of efgartigimod.

FIG. 14 is a graph showing the simulated maximum total IgG at day 29. The solid and dashed horizontal lines are the median and 90% CI of maximum total IgG reduction obtained with the 10 mg/kg IV weekly dose of efgartigimod. The points and bars are the median and 90% CI of total IgG reduction obtained with the SC weekly doses of efgartigimod.

FIG. 15 is a graph showing the percentage of simulated AUEC₂₂₋₂₉ (obtained with different efgartigimod PH20 SC weekly doses ranging between 750 mg and 1750 mg (with 25 mg increments)) greater than the median AUEC₂₂₋₂₉ obtained with 10 mg/kg IV of efgartigimod weekly.

FIG. 16 is a graph showing the percentage of simulated maximum total IgG reduction (IgGt supp) between day 22 and day 29 (obtained with different efgartigimod PH20 SC weekly doses ranging between 750 mg and 1750 mg (with 25 mg increments)) less than the median of the maximum total IgG reduction between day 22 and day 29 obtained with 10 mg/kg IV of efgartigimod weekly. Vertical dashed line: 975 mg; Horizontal dashed line: percentage obtained with 975 mg efgartigimod PH20 SC weekly.

FIG. 17 is a graph showing the percentage of simulated total IgG reduction (IgGt supp) on day 29 (trough) (obtained with different efgartigimod PH20 SC weekly doses ranging between 750 mg and 1750 mg (with 25 mg increments)) less than the median of total IgG reduction on day 29 obtained with 10 mg/kg IV of efgartigimod weekly.

FIG. 18 is a graph showing simulated AUEC in different time intervals obtained with 1000 mg efgartigimod PH20 SC weekly and 10 mg/kg efgartigimod IV weekly. Points and bars: median and 5^{th} and 95^{th} percentiles of AUEC.

FIG. 19 is a graph showing simulated maximum total IgG reduction in different time intervals obtained with 1000 mg efgartigimod PH20 SC weekly and 10 mg/kg efgartigimod IV weekly. Points and bars: median and 5^{th} and 95^{th} percentiles of maximum total IgG reduction.

FIG. 20 is a graph showing simulated total IgG reduction, before doses on days 8, 15, 22, and 29, obtained with 1000 mg efgartigimod PH20 SC weekly and 10 mg/kg efgartigimod IV weekly. Points and bars: median and 5^{th} and 95^{th} percentiles of total IgG reduction.

FIG. 21 is a graph showing simulated profiles of total IgG reduction after 1000 mg efgartigimod PH20 SC QW and 10 mg/kg IV of efgartigimod QW. Solid lines and areas: median, 5^{th} and 95^{th} percentiles of total IgG reduction; vertical dashed lines: day 22 and day 29.

FIG. 22 is a schematic of the clinical trial protocol for subcutaneous dosing of efgartigimod co-formulated with rHuPH20.

FIG. 23 is a graph showing mean (SE) of total IgG levels (μg/mL) over time during and after 4 weekly administrations of 1000 mg efgartigimod-PH20 SC or 10 mg/kg efgartigimod IV.

FIG. 24 is a graph showing mean (SE) percent change from baseline in total IgG over time during and after 4 weekly administrations of 1000 mg efgartigimod-PH20 SC or 10 mg/kg efgartigimod IV.

FIG. 25 is a graph showing mean difference and 95% 2-sided confidence intervals for the difference in change from baseline in total IgG between 4 weekly administrations of 1000 mg efgartigimod-PH20 SC and 10 mg/kg efgartigimod IV.

FIG. 26 is a graph showing mean (SD) efgartigimod serum concentration-time profiles after the fourth weekly administration of 1000 mg efgartigimod-PH20 SC or 10 mg/kg efgartigimod IV (day 22).

### DETAILED DESCRIPTION

The instant disclosure provides unit dosage forms of a biologic that are determined based on a modeling approach, which matches a pharmacodynamic (PD) value of the SC dose with that of a known reference IV dose, while a pharmacokinetic (PK) value of the SC dose is less than that of the IV dose. The unit dosage forms provided herein show comparable safety and efficacy as compared to a reference IV dose, and are therefore, non-inferior to the IV dose, providing patients with a more convenient alternative method of administration of a biologic.

Accordingly, provided herein are unit dosage forms for subcutaneous administration of a biologic, wherein the biologic has an RDᵢᵥ, which results in a PKᵢᵥ and a PDᵢᵥ in a subject upon intravenous administration; the unit dosage form comprises an RD_{sc} of the biologic, which results in a PK_{sc} and a PD_{sc} in a subject upon subcutaneous administration; and the ratio PK_{sc}/PKᵢᵥ is less than 0.8 and the ratio PD_{sc}/PDᵢᵥ is from 0.9 to 1.1.

### Definitions

As used herein, the term unit dosage form is a pharmaceutical drug product in the form in which it is marketed for use, with a specific mixture of active ingredients and inactive components (excipients), and apportioned into a particular dose. Unit dosage forms provided herein can refer to physically discrete units suitable as unitary dosages for human and/or animal subjects, each unit containing a predetermined quantity of active material (e.g., about 500 mg to about 2500 mg of efgartigimod or about 500 mg to about 2500 mg of efgartigimod and about 1000 U/ml to about 3000 U/ml rHuPH20) calculated to produce the desired therapeutic effect in association with the required pharmaceutical diluent, carrier, or vehicle. Non-limiting examples of suitable unit dosage forms are vials, tablets, capsules, troches, suppositories, powder packets, wafers, cachets, ampules, pre-filled syringes, segregated multiples of any of the foregoing, and other forms as herein described or generally known in the art.

As used herein, the term “biologic” refers to a product that is produced from living organisms or contain components of living organisms, for example antibodies or antibody fragments or recombinant proteins. In an embodiment, the biologic is efgartigimod.

As used herein, the term “reference dose” refers to an arbitrary intravenous dose of the biologic of which the PK and/or PD (PKᵢᵥ and PDᵢᵥ) are used as reference values. In an embodiment, the reference dose may be an approved drug dose, a specific determined drug dose, or an optimal drug dose as determined during clinical trial(s). In an embodiment, the reference dose of the biologic can be the approved dose by a regulatory authority (such as the Food and Drug Administration (FDA) in US or European Medicines Agency (EMA) in Europa) for administration to a patient.

As used herein, the term “RDᵢᵥ” refers to a dose of a biologic for intravenous administration to a patient, generally in one single administration.

As used herein, the term “RD_{sc}” refers to a dose of a biologic for subcutaneous administration to a patient, generally in one single administration.

As used herein, the term “PKᵢᵥ” refers to an experimentally determined pharmacokinetic value for an intravenously administered drug. This value is used to describe the absorption, distribution, metabolism, and excretion of the drug in the (human) body.

As used herein, the term “PK_{sc}” refers to a pharmacokinetic value for a subcutaneously administered drug. This value is used to describe the absorption, distribution, metabolism, and excretion of the drug in the (human) body. In an embodiment, a PK_{sc} can be determined based on pharmacokinetic modeling (predictive modeling methods). In an embodiment, a PK_{sc} can be determined experimentally or empirically (e.g., based on experience).

As used herein, the term “PDᵢᵥ” refers to an experimentally determined pharmacodynamic value of an intravenously administered drug. In an embodiment, this value is used to describe the biochemical, physiologic, and molecular effects (clinical effects) of the drug on the (human) body and involves receptor binding (including receptor sensitivity), post receptor effects, and chemical interactions.

As used herein, the term “PD_{sc}” refers to a pharmacodynamic value of a subcutaneously administered drug. In an embodiment, this value is used to describe the biochemical, physiologic, and molecular effects (clinical effects) of the drug on the (human) body and involves receptor binding (including receptor sensitivity), post receptor effects, and chemical interactions. In an embodiment, a PD_{sc} can be determined based on pharmacodynamic modeling (predictive modeling methods). In an embodiment, PD_{sc} can be determined experimentally or empirically (e.g., based on experience).

As used herein, the term “BLᵢᵥ” refers to the level of a biomarker (e.g., IgG) following intravenous administration of a biologic to a subject, compared to a baseline level of the biomarker in the subject.

As used herein, the term “BL_{sc}” refers to the level of a biomarker (e.g., IgG) following subcutaneous administration of a biologic to a subject, compared to a baseline level of the biomarker in the subject.

As used herein, the term “Cₘₐₓ” refers to the maximum serum concentration of a biologic.

As used herein, the term “AUC” refers to the area under the serum concentration versus time curve. The AUC is based on the rate and extent of elimination of a biologic following administration.

As used herein, the term “Fc domain” refers to the portion of a single immunoglobulin heavy chain beginning in the hinge region and ending at the C-terminus of the antibody. Accordingly, a complete Fc domain comprises at least a portion of a hinge (e.g., upper, middle, and/or lower hinge region) domain, a CH2 domain, and a CH3 domain.

As used herein, the term “Fc region” refers to the portion of a native immunoglobulin formed by the Fc domains of its two heavy chains. A native Fc region is homodimeric.

As used herein, the term “variant Fc region” refers to an Fc region with one or more alteration(s) relative to a native Fc region. Alteration can include amino acid substitutions, additions and/or deletions, linkage of additional moieties, and/or alteration the native glycans. The term encompasses heterodimeric Fc regions where each of the constituent Fc domains is different. The term also encompasses single chain Fc regions where the constituent Fc domains are linked together by a linker moiety.

As used herein the term “FcRn binding fragment” refers to a portion of an Fc region that is sufficient to confer FcRn binding.

As used herein, the term “hyaluronidase enzyme” refers to an enzyme that catalyzes the breakdown of hyaluronic acid in the body, which may increase the permeability of tissue to fluids or drugs (e.g., a subcutaneously administered biologic). In an embodiment, the hyaluronidase enzyme is a recombinant human hyaluronidase PH20 enzyme (rHuPH20) which degrades hyaluronan (HA).

As used herein, the term “IgG reduction” refers to a decline of (disease-causing) immunoglobulin G (IgG) antibodies, e.g., in a patient's blood serum.

As used herein, the term “baseline IgG level” refers to the IgG level in a patient, e.g., in a patient's blood, prior to the first administration (e.g., intravenous, or subcutaneous administration) of a biologic.

As used herein, the term “bioanalytical method” refers to a bioanalytical assay that is used for the quantification of molecules (e.g., proteins, antibodies such as IgGs, and therapeutic agents) in support of pharmacokinetic evaluations, for example, to measure the total IgG in a serum sample. In an embodiment, the bioanalytical method is an ELISA. In an embodiment, the bioanalytical method is an automated diagnostic analyzer (IVD).

As used herein, the term “about” or “approximately” when referring to a measurable value, such as a dosage, encompasses variations of ±20% or ±10%, ±5%, ±1%, or ±0.10% of a given value or range, as are appropriate to perform the methods disclosed herein.

### Subcutaneous Unit Dosage Form Compositions and Methods

The instant disclosure provides unit dosage forms of a biologic for subcutaneous administration to a subject. These unit dosage forms comprise an effective amount of a biologic, wherein the effective amount is determined based on a modeling approach, which matches a pharmacodynamic (PD) value of the SC dose with that of a known reference IV dose, while a pharmacokinetic (PK) value of the SC dose is less than that of the IV dose. The unit dosage forms provided herein show comparable safety and efficacy as compared to a reference IV dose, and are therefore, non-inferior to the IV dose, providing patients with a more convenient alternative method of administration of a biologic.

Previously known methods of determining an SC dose are based on models that aim to match a PK value of an SC dose and a reference IV dose, which leads to unit dosage forms with higher dosage amounts of biologics, as compared to the methods used herein. Accordingly, the unit dosage forms disclosed herein comprise lower dosage amounts of a biologic, which could decrease adverse events in patients and could allow for subcutaneous administration as an alternative for biologics that are generally administered by IV infusion.

Accordingly, provided herein are unit dosage forms for subcutaneous administration of a biologic, wherein the biologic has an RDᵢᵥ, which results in a PKᵢᵥ and a PDᵢᵥ in a subject upon intravenous administration; the unit dosage form comprises an RD_{sc} of the biologic, which results in a PK_{sc} and a PD_{sc} in a subject upon subcutaneous administration; and the ratio PK_{sc}/PKᵢᵥ is less than 0.8 and the ratio PD_{sc}/PDᵢᵥ is from 0.9 to 1.1.

Also provided herein are unit dosage forms for subcutaneous administration of a biologic, wherein the biologic has an RDᵢᵥ, which results in a PKᵢᵥ and a BLᵢᵥ in a subject upon intravenous administration, the unit dosage form comprises an RD_{sc} of the biologic, which results in a PK_{sc} and a BL_{sc} in a subject upon subcutaneous administration; and the ratio PK_{sc}/PKᵢᵥ is less than about 0.8 and the ratio BL_{sc}/BLᵢᵥ is of about 0.9 to about 1.1.

Also provided herein are unit dosage forms for subcutaneous administration of a biologic, wherein the amount subcutaneous dose of the biologic in the unit dosage form was determined by a method comprising the steps of (a) administering a subcutaneous dose of the biologic to a subject, wherein the biologic has an RDᵢᵥ, which results in a PKᵢᵥ and a BLᵢᵥ; (b) determining the BL_{sc}; (c) determining the PK_{sc} of the biologic; and (d) determining a subcutaneous dose that would result in a BL_{sc}/BLᵢᵥ ratio of about 0.9 to about 1.1 and a PK_{sc}/PKᵢᵥ ratio less than about 0.8.

Also provided herein is method of determining a therapeutically effective dose of a biologic for subcutaneous administration, the method comprising: (a) administering a subcutaneous dose of the biologic to a subject, wherein the biologic has an RDᵢᵥ, which results in a PKᵢᵥ and a BLᵢᵥ; (b) determining the BL_{sc} of the biologic; (c) determining the PK_{sc} of the biologic; and (d) determining a subcutaneous dose that would result in a BL_{sc}/BLᵢᵥ ratio of about 0.9 to about 1.1 and a PK_{sc}/PKᵢᵥ ratio less than about 0.8, thereby determining a therapeutically effective dose of the biologic for subcutaneous administration.

In an embodiment, the subject is a healthy volunteer or a non-human animal.

Also provided herein is a method of treating a subject with a subcutaneous dose of a biologic, wherein the subcutaneous dose of the biologic was determined by a method comprising the steps of: (a) administering a subcutaneous dose of the biologic to a subject, wherein the biologic has an RDᵢᵥ, which results in a PKᵢᵥ and a BLᵢᵥ; (b) determining the BL_{sc} of the biologic; (c) determining the PK_{sc} of the biologic; and (d) determining a subcutaneous dose that would result in a BL_{sc}/BLᵢᵥ ratio of about 0.9 to about 1.1 and a PK_{sc}/PKᵢᵥ ratio less than about 0.8.

In an embodiment, the ratio PK_{sc}/PKᵢᵥ is less than 0.7. In an embodiment, the ratio PK_{sc}/PKᵢᵥ is less than 0.6. In an embodiment, the PKᵢᵥ and the PK_{sc} values are the AUC.

In an embodiment, the BL_{sc} and the BLᵢᵥ are levels of total serum IgG the subject. In an embodiment, the total serum IgG in the subject is analyzed using a bioanalytical method. In an embodiment, the bioanalytical method is ELISA or automated diagnostic analyzer (IVD).

In an embodiment, the biologic is an antibody molecule. In an embodiment, the antibody molecule binds FcRn. In an embodiment, the antibody molecule comprises an Fc domain engineered for optimized binding to FcRn. In an embodiment, the antibody molecule blocks FcRn.

In an embodiment, the biologic is a variant Fc region, or FcRn binding fragment thereof. In an embodiment, the biologic is efgartigimod.

In an embodiment, the biologic is selected from the group consisting of antibodies, antibody fragments, anticoagulants, blood factors, bone morphogenetic proteins, enzymes, fusion proteins, growth factors, hormones, interferons, interleukins, and thrombolytics.

In an embodiment, the PK_{sc}/PKᵢᵥ ratio is less than 0.7. In an embodiment, the PK_{sc}/PKᵢᵥ ratio is less than 0.6. In an embodiment, the PK_{sc}/PKᵢᵥ ratio is about 0.8, about 0.7, about 0.6, about 0.5, about 0.47, or about 0.4. In an embodiment, the PK_{sc}/PKᵢᵥ ratio is about 0.8. In an embodiment, the PK_{sc}/PKᵢᵥ ratio is about 0.7. In an embodiment, the PK_{sc}/PKᵢᵥ ratio is about 0.6. In an embodiment, the PK_{sc}/PKᵢᵥ ratio is about 0.5. In an embodiment, the PK_{sc}/PKᵢᵥ ratio is about 0.4.

In an embodiment, the PD_{sc}/PDᵢᵥ ratio is from 0.9 to 1.1. In an embodiment, the PD_{sc}/PDᵢᵥ ratio is 0.9, 1.0, or 1.1. In an embodiment, the PD_{sc}/PDᵢᵥ ratio is about 0.9, about 0.91, about 0.92, about 0.93, about 0.94, about 0.95, about 0.96, about 0.97, about 0.98, or about 0.99. In an embodiment, the PD_{sc}/PDᵢᵥ ratio is about 1.0, about 1.01, about 1.02, about 1.03, about 1.04, about 1.05, about 1.06, about 1.07, about 1.08, or about 1.09. In an embodiment, the PD_{sc}/PDᵢᵥ ratio is about 1.1, about 1.11, about 1.12, about 1.13, about 1.14, about 1.15, about 1.16, about 1.17, about 1.18, or about 1.19.

In an embodiment, the BL_{sc}/BLᵢᵥ ratio is from 0.9 to 1.1. In an embodiment, the BLₛₑ/BLᵢᵥ ratio is 0.9, 1.0, or 1.1. In an embodiment, the BL_{sc}/BLᵢᵥ ratio is about 0.9, about 0.91, about 0.92, about 0.93, about 0.94, about 0.95, about 0.96, about 0.97, about 0.98, or about 0.99. In an embodiment, the BL_{sc}/BLᵢᵥ ratio is about 1.0, about 1.01, about 1.02, about 1.03, about 1.04, about 1.05, about 1.06, about 1.07, about 1.08, or about 1.09. In an embodiment, the BL_{sc}/BLᵢᵥ ratio is about 1.1, about 1.11, about 1.12, about 1.13, about 1.14, about 1.15, about 1.16, about 1.17, about 1.18, or about 1.19.

In an embodiment, the PK_{sc}/PKᵢᵥ ratio is less than 0.8 and the PD_{sc}/PDᵢᵥ ratio is about 0.9. In an embodiment, the PK_{sc}/PKᵢᵥ ratio is less than 0.7 and the PD_{sc}/PDᵢᵥ ratio is about 0.9. In an embodiment, the PK_{sc}/PKᵢᵥ ratio is less than 0.6 and the PD_{sc}/PDᵢᵥ ratio is about 0.9. In an embodiment, the PK_{sc}/PKᵢᵥ ratio is about 0.7 and the PD_{sc}/PDᵢᵥ ratio is about 0.9. In an embodiment, the PK_{sc}/PKᵢᵥ ratio is about 0.6 and the PD_{sc}/PDᵢᵥ ratio is about 0.9. In an embodiment, the PK_{sc}/PKᵢᵥ ratio is about 0.5 and the PD_{sc}/PDᵢᵥ ratio is about 0.9. In an embodiment, the PK_{sc}/PKᵢᵥ ratio is about 0.4 and the PD_{sc}/PDᵢᵥ ratio is about 0.9.

In an embodiment, the PK_{sc}/PKᵢᵥ ratio is less than 0.8 and the PD_{sc}/PDᵢᵥ ratio is about 1.0. In an embodiment, the PK_{sc}/PKᵢᵥ ratio is less than 0.7 and the PD_{sc}/PDᵢᵥ ratio is about 1.0. In an embodiment, the PK_{sc}/PKᵢᵥ ratio is less than 0.6 and the PD_{sc}/PDᵢᵥ ratio is about 1.0. In an embodiment, the PK_{sc}/PKᵢᵥ ratio is about 0.7 and the PD_{sc}/PDᵢᵥ ratio is about 1.0. In an embodiment, the PK_{sc}/PKᵢᵥ ratio is about 0.6 and the PD_{sc}/PDᵢᵥ ratio is about 1.0. In an embodiment, the PK_{sc}/PKᵢᵥ ratio is about 0.5 and the PD_{sc}/PDᵢᵥ ratio is about 1.0. In an embodiment, the PK_{sc}/PKᵢᵥ ratio is about 0.4 and the PD_{sc}/PDᵢᵥ ratio is about 1.0.

In an embodiment, the PK_{sc}/PKᵢᵥ ratio is less than 0.8 and the PD_{sc}/PDᵢᵥ ratio is about 1.1. In an embodiment, the PK_{sc}/PKᵢᵥ ratio is less than 0.7 and the PD_{sc}/PDᵢᵥ ratio is about 1.1. In an embodiment, the PK_{sc}/PKᵢᵥ ratio is less than 0.6 and the PD_{sc}/PDᵢᵥ ratio is about 1.1. In an embodiment, the PK_{sc}/PKᵢᵥ ratio is about 0.7 and the PD_{sc}/PDᵢᵥ ratio is about 1.1. In an embodiment, the PK_{sc}/PKᵢᵥ ratio is about 0.6 and the PD_{sc}/PDᵢᵥ ratio is about 1.1. In an embodiment, the PK_{sc}/PKᵢᵥ ratio is about 0.5 and the PD_{sc}/PDᵢᵥ ratio is about 1.1. In an embodiment, the PK_{sc}/PKᵢᵥ ratio is about 0.4 and the PD_{sc}/PDᵢᵥ ratio is about 1.1.

In an embodiment, the PK_{sc}/PKᵢᵥ ratio is less than 0.8 and the PD_{sc}/PDᵢᵥ ratio is about 0.9, about 0.91, about 0.92, about 0.93, about 0.94, about 0.95, about 0.96, about 0.97, about 0.98, or about 0.99. In an embodiment, the PK_{sc}/PKᵢᵥ ratio is less than 0.8 and the PD_{sc}/PDᵢᵥ ratio is about 1.0, about 1.01, about 1.02, about 1.03, about 1.04, about 1.05, about 1.06, about 1.07, about 1.08, or about 1.09. In an embodiment, the PK_{sc}/PKᵢᵥ ratio is less than 0.8 and the PD_{sc}/PDᵢᵥ ratio is about 1.1, about 1.11, about 1.12, about 1.13, about 1.14, about 1.15, about 1.16, about 1.17, about 1.18, or about 1.19.

In an embodiment, the PK_{sc}/PKᵢᵥ ratio is less than 0.7 and the PD_{sc}/PDᵢᵥ ratio is about 0.9, about 0.91, about 0.92, about 0.93, about 0.94, about 0.95, about 0.96, about 0.97, about 0.98, or about 0.99. In an embodiment, the PK_{sc}/PKᵢᵥ ratio is less than 0.7 and the PD_{sc}/PDᵢᵥ ratio is about 1.0, about 1.01, about 1.02, about 1.03, about 1.04, about 1.05, about 1.06, about 1.07, about 1.08, or about 1.09. In an embodiment, the PK_{sc}/PKᵢᵥ ratio is less than 0.7 and the PD_{sc}/PDᵢᵥ ratio is about 1.1, about 1.11, about 1.12, about 1.13, about 1.14, about 1.15, about 1.16, about 1.17, about 1.18, or about 1.19.

In an embodiment, the PK_{sc}/PKᵢᵥ ratio is less than 0.6 and the PD_{sc}/PDᵢᵥ ratio is about 0.9, about 0.91, about 0.92, about 0.93, about 0.94, about 0.95, about 0.96, about 0.97, about 0.98, or about 0.99. In an embodiment, the PK_{sc}/PKᵢᵥ ratio is less than 0.6 and the PD_{sc}/PDᵢᵥ ratio is about 1.0, about 1.01, about 1.02, about 1.03, about 1.04, about 1.05, about 1.06, about 1.07, about 1.08, or about 1.09. In an embodiment, the PK_{sc}/PKᵢᵥ ratio is less than 0.6 and the PD_{sc}/PDᵢᵥ ratio is about 1.1, about 1.11, about 1.12, about 1.13, about 1.14, about 1.15, about 1.16, about 1.17, about 1.18, or about 1.19.

In an embodiment, the RDᵢᵥ is from 10 mg/kg to 25 mg/kg and the RD_{sc} is from about 1000 mg to about 2000 mg. In an embodiment, the RDᵢᵥ is 10 mg/kg and the RD_{sc} is about 1000 mg. In an embodiment, the RDᵢᵥ is 25 mg/kg and the RD_{sc} is about 2000 mg. In an embodiment, the RDᵢᵥ is 10 mg/kg and the RD_{sc} is about 2000 mg. In an embodiment, the RDᵢᵥ is 25 mg/kg and the RD_{sc} is about 1000 mg. In an embodiment, the RDᵢᵥ is about 10 mg/kg to about 15 mg/kg and the RD_{sc} is about 1000 mg to about 1500 mg. In an embodiment, the RDᵢᵥ is 20 mg/kg to about 25 mg/kg and the RD_{sc} is about 1500 mg to about 2000 mg.

In an embodiment, the PKᵢᵥ and the PK_{sc} values are the AUC. In an embodiment, the PDᵢᵥ and the PD_{sc} values are the total serum IgG reduction in the subject.

In an embodiment, the unit dosage form further comprises a hyaluronidase enzyme. In an embodiment, the hyaluronidase enzyme is rHuPH20.

In an embodiment, the unit dosage form is co-administered with a hyaluronidase enzyme. In an embodiment, the hyaluronidase enzyme is rHuPH20.

In an embodiment, the amount of hyaluronidase enzyme is from about 1000 U/ml to about 3000 U/ml. In an embodiment, the amount of hyaluronidase enzyme is about 1000 U/mL, about 1500 U/mL, about 2000 U/mL, about 2500 U/mL, or about 3000 U/mL. In an embodiment, the amount of hyaluronidase enzyme is 2000 U/mL.

In an embodiment, the unit dosage form comprises from about 1000 U/ml to about 3000 U/ml of rHuPH20. In an embodiment, the unit dosage form comprises about 1000 U/mL, about 1500 U/mL, about 2000 U/mL, about 2500 U/mL, or about 3000 U/mL of rHuPH20. In an embodiment, the unit dosage form comprises 1000 U/mL of rHuPH20. In an embodiment, the unit dosage form comprises 1500 U/mL of rHuPH20. In an embodiment, the unit dosage form comprises 2000 U/mL of rHuPH20. In an embodiment, the unit dosage form comprises 2500 U/mL of rHuPH20. In an embodiment, the unit dosage form comprises 3000 U/mL of rHuPH20.

In an embodiment, the biologic is an antibody molecule. In an embodiment, the antibody molecule comprises an Fc domain engineered for optimized binding to FcRn. In an embodiment, the antibody molecule blocks FcRn. In an embodiment, the biologic is efgartigimod.

In an embodiment, the unit dosage form comprises about 500 mg to about 2500 mg of efgartigimod. In an embodiment, the unit dosage form comprises about 500 mg to about 1000 mg of efgartigimod. In an embodiment, the unit dosage form comprises about 1000 mg to about 1500 mg of efgartigimod. In an embodiment, the unit dosage form comprises about 1500 mg to about 2000 mg of efgartigimod. In an embodiment, the unit dosage form comprises about 1500 mg to about 2000 mg of efgartigimod.

In an embodiment, the unit dosage form comprises about 500 mg of efgartigimod. In an embodiment, the unit dosage form comprises about 750 mg of efgartigimod. In an embodiment, the unit dosage form comprises about 1000 mg of efgartigimod. In an embodiment, the unit dosage form comprises about 1250 mg of efgartigimod. In an embodiment, the unit dosage form comprises about 1500 mg of efgartigimod. In an embodiment, the unit dosage form comprises about 1750 mg of efgartigimod. In an embodiment, the unit dosage form comprises about 2000 mg of efgartigimod. In an embodiment, the unit dosage form comprises about 2250 mg of efgartigimod. In an embodiment, the unit dosage form comprises about 2500 mg of efgartigimod.

In an embodiment, the unit dosage form comprises about 500 mg of efgartigimod and about 2000 U/mL of rHuPH20. In an embodiment, the unit dosage form comprises about 750 mg of efgartigimod and about 2000 U/mL of rHuPH20. In an embodiment, the unit dosage form comprises about 1000 mg of efgartigimod and about 2000 U/mL of rHuPH20. In an embodiment, the unit dosage form comprises about 1250 mg of efgartigimod and about 2000 U/mL of rHuPH20. In an embodiment, the unit dosage form comprises about 1500 mg of efgartigimod and about 2000 U/mL of rHuPH20. In an embodiment, the unit dosage form comprises about 1750 mg of efgartigimod and about 2000 U/mL of rHuPH20. In an embodiment, the unit dosage form comprises about 2000 mg of efgartigimod and about 2000 U/mL of rHuPH20. In an embodiment, the unit dosage form comprises about 2250 mg of efgartigimod and about 2000 U/mL of rHuPH20. In an embodiment, the unit dosage form comprises about 2500 mg of efgartigimod and about 2000 U/mL of rHuPH20.

In an embodiment, the unit dosage form comprises about 500 mg of efgartigimod and about 1000 U/mL to about 3000 U/mL of rHuPH20. In an embodiment, the unit dosage form comprises about 750 mg of efgartigimod and about 1000 U/mL to about 3000 U/mL of rHuPH20. In an embodiment, the unit dosage form comprises about 1000 mg of efgartigimod and about 1000 U/mL to about 3000 U/mL of rHuPH20. In an embodiment, the unit dosage form comprises about 1250 mg of efgartigimod and about 1000 U/mL to about 3000 U/mL of rHuPH20. In an embodiment, the unit dosage form comprises about 1500 mg of efgartigimod and about 1000 U/mL to about 3000 U/mL of rHuPH20. In an embodiment, the unit dosage form comprises about 1750 mg of efgartigimod and about 1000 U/mL to about 3000 U/mL of rHuPH20. In an embodiment, the unit dosage form comprises about 2000 mg of efgartigimod and about 1000 U/mL to about 3000 U/mL of rHuPH20. In an embodiment, the unit dosage form comprises about 2250 mg of efgartigimod and about 1000 U/mL to about 3000 U/mL of rHuPH20. In an embodiment, the unit dosage form comprises about 2500 mg of efgartigimod and about 1000 U/mL to about 3000 U/mL of rHuPH20.

In an embodiment, the unit dosage form comprises the antibody molecule as a dry formulation for dissolution such as a lyophilized powder, freeze-dried powder, or water free concentrate. In an embodiment, the dry formulation is comprised in a hermetically sealed container such as a vial, an ampoule, or a sachet.

In an embodiment, the unit dosage form comprises the antibody molecule as a liquid formulation, e.g., injection or infusion solution. In an embodiment, the liquid formulation is comprised in a hermetically sealed container such as a vial, a sachet, a pre-filled syringe, a pre-filled autoinjector, or a cartridge for a reusable syringe or applicator.

In an embodiment, the unit dosage per vial may contain 0.5 ml, 1 ml, 2 ml, 3 ml, 4 ml, 5 ml, 6 ml, 7 ml, 8 ml, 9 ml, 10 ml, 15 ml, or 20 ml of an antibody molecule ranging from about 500 to about 2500 mg or from about 1000 mg to about 2000 mg. In an embodiment, these preparations can be adjusted to a desired concentration by adding a sterile diluent to each vial.

The formulations disclosed herein include bulk drug compositions useful in the manufacture of pharmaceutical compositions (e.g., compositions that are suitable for administration to a subject or patient) which can be used in the preparation of unit dosage forms. In an embodiment, a composition of the invention is a pharmaceutical composition. Such compositions comprise a prophylactically or therapeutically effective amount of one or more prophylactic or therapeutic agents (e.g., an antibody molecule of the invention or other prophylactic or therapeutic agent), and a pharmaceutically acceptable carrier. In an embodiment, the pharmaceutical compositions are formulated to be suitable for subcutaneous administration to a subject.

### Soluble Hyaluronidases

Provided in the co-formulations, unit dosage forms, and methods herein are soluble hyaluronidases. Soluble hyaluronidases include any, that, upon expression and secretion from a cell, exist in soluble form. Such soluble hyaluronidases include, but are not limited to, non-human soluble hyaluronidases, bacterial soluble hyaluronidases, bovine PH20, ovine PH20, and variants thereof. Included among the soluble hyaluronidases are human PH20 polypeptides that have been modified to be soluble. For example, hyaluronidases, such as human PH20, that contain a glycophophatidylinositoal (GPI) anchor can be made soluble by truncation of and removal of all or a portion of the GPI anchor. In an embodiment, the human hyaluronidase PH20, which is normally membrane anchored via a GPI anchor, is made soluble by truncation of and removal of all or a portion of the GPI anchor at the C-terminus.

Soluble hyaluronidases also include neutral active hyaluronidases, such as the soluble human PH20 polypeptides. In an embodiment, the hyaluronidase for use in the compositions, unit dosage forms, and methods herein is a soluble neutral active hyaluronidase.

Exemplary hyaluronidases include a soluble form of a PH20 from any species, such as a soluble form of a PH20 of any of SEQ ID NOs: 5-40, and such as the soluble PH20 polypeptides set forth in SEQ ID NOs. 5 and 18-23. Such soluble forms include truncated forms thereof lacking all or a portion of the C-terminal GPI anchor, so long as the hyaluronidase is soluble (secreted upon expression) and retains hyaluronidase activity. Such forms also typically are mature forms that, when expressed in a cell, lack the signal peptide. Also included among soluble hyaluronidases are soluble forms of variants of any of the PH20s from any species set forth in SEQ ID NOs: 5-40 that exhibit hyaluronidase activity. Variants include polypeptides having at least 60%, 70%, 80%, 85%, 90%, 91%, 92%, 93%, 94%, 95%, 96%, 97%, 98%, 99% or more sequence identity to any of SEQ ID NOs: 5-40. Amino acid variants include conservative and non-conservative mutations. It is understood that residues that are important or otherwise required for the activity of a hyaluronidase, such as any described above or known to skill in the art, are generally invariant and cannot be changed. These include, for example, active site residues. Thus, for example, amino acid residues 111, 113 and 176 (corresponding to residues in the mature PH20 polypeptide set forth in SEQ ID NO: 5) of a human PH20 polypeptide, or soluble form thereof, are generally invariant and are not altered. Other residues that confer glycosylation and formation of disulfide bonds required for proper folding also can be invariant.

In an embodiment, the soluble hyaluronidase is normally GPI-anchored (such as, for example, human PH20) and is rendered soluble by truncation at the C-terminus. Such truncation can remove all of the GPI anchor attachment signal sequence, or can remove only some of the GPI anchor attachment signal sequence. The resulting polypeptide, however, is soluble. In instances where the soluble hyaluronidase retains a portion of the GPI anchor attachment signal sequence, 1, 2, 3, 4, 5, 6, 7 or more amino acid residues in the GPI anchor attachment signal sequence can be retained, provided the polypeptide is soluble. Polypeptides containing one or more amino acids of the GPI anchor are termed extended soluble hyaluronidases. One of skill in the art can determine whether a polypeptide is GPI-anchored using methods well known in the art. Such methods include, but are not limited to, using known algorithms to predict the presence and location of the GPI anchor attachment signal sequence and ω-site, and performing solubility analyses before and after digestion with phosphatidylinositol-specific phospholipase C (PI-PLC) or D (PI-PLD).

Extended soluble hyaluronidases, such as those set forth in SEQ ID NOs: 42-47, can be produced by making C-terminal truncations to any naturally GPI-anchored hyaluronidase such that the resulting polypeptide is soluble and contains one or more amino acid residues from the GPI anchor attachment signal sequence (see, e.g., U.S. Pat. No. 8,927,249). These include hyaluronidases that are neutral active, soluble, contain amino acid substitutions, and have at least 60%, 70%, 80%, 85%, 90%, 91%, 92%, 93%, 94%, 95% or more sequence identity to any of SEQ ID NOs: 42-47.

Typically, for use in the compositions, combinations and methods herein, a soluble human hyaluronidase, such as a soluble human PH20, is used, such as a PH20 polypeptide of any of SEQ ID NOs: 5 and 18-23 and variants having, for example, at least 98% sequence identity thereto. Hyaluronidases used in the methods herein can be recombinantly produced or can be purified or partially-purified from natural sources, such as, for example, from testes extracts. Methods for production of recombinant proteins, including recombinant hyaluronidases, are well known in the art.

### (a) Soluble Human PH20

An exemplary soluble hyaluronidase is soluble human PH20. Soluble forms of recombinant human PH20 have been produced and can be used in the compositions, combinations and methods described herein. The description of and production of such soluble forms of PH20 is described, for example, in U.S. Pat. Nos. 7,767,429, 8,202,517, 8,431,380, 8,431,124, 8,450,470 8,765,685, 8,772,246, 7,871,607, 7,846,431, 7,829,081, 8,105,586, 8,187,855, 8,257,699, 8,580,252, 9,677,061, and 9,677,062 which are incorporated by reference herein.

Recombinant soluble forms of human PH20 have been generated and can be used in the compositions, combinations and methods provided herein. For example, with reference to SEQ ID NO: 4, which sets forth the sequence of full length precursor PH20, which includes a signal sequence (residues 1-35), soluble forms include, but are not limited to, C-terminal truncated polypeptides of human PH20 set forth in SEQ ID NO: 4 having a C-terminal amino acid residue 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO: 4, or polypeptides that exhibit at least 85%, 90%, 91%, 92%, 93%, 94%, 95%, 96%, 97%, 98%, 99%, or more sequence identity thereto, have activity at neutral pH, and are soluble (secreted into the medium when expressed in a mammalian cell). Soluble forms of human PH20 generally include those that contain amino acids 36-464 set forth in SEQ ID NO: 4. For example, when expressed in mammalian cells, the 35 amino acid N-terminal signal sequence is cleaved during processing, and the mature form of the protein is secreted. Thus, the mature soluble polypeptides include those that contain amino acids 36 to 467, 468, 469, 470, 471, 472, 473, 474, 475, 476, 477, 478, 479, 480, 481, 482 and 483 of SEQ ID NO: 4. In an embodiment, soluble hyaluronidases are soluble human PH20 polypeptides that are 442, 443, 444, 445, 446 or 447 amino acids in length, such as set forth in any of SEQ ID NOs: 5 and 18-23 and variants thereof that have, for example, at least 85%, 86%, 87%, 88%, 89%, 90%, 91%, 92%, 93%, 94%, 95%, 96%, 97%, 98% or 99% sequence identity to a sequence of amino acids set forth in any of SEQ ID NOs: 5 and 18-23 and retains hyaluronidase activity. The generation of such soluble forms of recombinant human PH20 are described, for example, in U.S. Pat. Nos. 7,767,429, 8,202,517, 8,431,380, 8,431,124, 8,450,470 8,765,685, 8,772,246, 7,871,607, 7,846,431, 7,829,081, 8,105,586, 8,187,855, 8,257,699, 8,580,252, 9,677,061, and 9,677,062.

Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g. DG44 CHO cells).

(b) rHuPH20

rHuPH20 refers to the composition produced upon expression in a cell, such as a CHO cell, of nucleic acid encoding residues 36-482 of SEQ ID NO: 4, generally linked to the native or a heterologous signal sequence (residues 1-35 of SEQ ID NO: 4). rHuPH20 is produced by expression of a nucleic acid molecule, such as encoding amino acids 1-482 (set forth in SEQ ID NO: 4). Post translational processing removes the 35 amino acid signal sequence, leaving a polypeptide or a mixture of polypetides, including those set forth in SEQ ID NOs: 5 and 18-23. As produced in the culture medium there is heterogeneity at the C-terminus such that the product, designated rHuPH20, includes a mixture of species that can include any one or more of SEQ ID NOs: 5 and 18-23 in various abundance. Typically, rHuPH20 is produced in cells that facilitate correct N-glycosylation to retain activity, such as CHO cells (e.g. DG44 CHO cells). Generally the most abundant species is the 446 amino acid polypeptide corresponding to residues 36-481 of SEQ ID NO: 4.

### (c) Glycosylation of Hyaluronidases

Glycosylation, including N- and O-linked glycosylation, of some hyaluronidases, including the soluble PH20 hyaluronidases, can be important for their catalytic activity and stability. For some hyaluronidases, removal of N-linked glycosylation can result in near complete inactivation of the hyaluronidase activity. Thus, for such hyaluronidases, the presence of N-linked glycans can be important for generating an active enzyme.

N-linked oligosaccharides fall into several primary types (oligomannose, complex, hybrid, sulfated), all of which have (Man) 3-GlcNAc-GlcNAc-cores attached via the amide nitrogen of Asn residues that fall within-Asn-Xaa-Thr/Ser-sequences (where Xaa is not Pro). Glycosylation at an-Asn-Xaa-Cys-site has been reported for coagulation protein C. In some instances, a hyaluronidase, such as a PH20 hyaluronidase, can contain N-glycosidic and O-glycosidic linkages. For example, PH20 has O-linked oligosaccharides as well as N-linked oligosaccharides. There are six potential N-linked glycosylation sites at N82, N166, N235, N254, N368, N393 of human PH20 exemplified in SEQ ID NO: 1.

### (d) Variants

Variants of the soluble PH20 polypeptides that have altered properties, such as increased stability and/or activity, have been produced. U.S. Pat. Nos. 9,447,401 and 10,865,400, and allowed application Ser. No. 16/824,572, which are incorporated by reference, describe and provide a structure/function map of human PH20 detailing the effects of amino acid replacements at every residue in the catalytic domain of PH20. Theses patents provide about 7000 examples in which the effects of replacing each amino acid with 15 other amino acids on activity and stability were identified and described. Most variants of soluble PH20 polypeptides, including those with amino acid replacements, deletions, and insertions, are known in the art. A skilled person readily can prepare soluble hyaluronidases and variants thereof and know the properties of the resulting hyaluronidase.

Other variants known to those of skill in the art are described in International PCT application Nos. WO2020/022791 and WO2020197230A, which are incorporated by reference, and which describe modified PH20 polypeptides. These polypeptides, which are variants of the PH20 polypeptides of SEQ ID NOs: 5-40 include replacements, insertions, and deletions, including one or more amino acid residues S343E, M345T, K349E, L353A, L354I, N356E, and 1361T. Variants that contain such modifications and others are set forth in SEQ ID NOs: 41-96 from International PCT application No WO2020/022791.

### Biologics

Provided herein are unit dosage forms of a biologic that are determined based on a modeling approach, which matches a pharmacodynamic (PD) value of the SC dose with that of a known reference IV dose, while a pharmacokinetic (PK) value of the SC dose is less than that of the IV dose. The unit dosage forms provided herein show comparable safety and efficacy as compared to a reference IV dose, and are therefore, non-inferior to the IV dose, providing patients with a more convenient alternative method of administration of a biologic.

Non-limiting examples of biologics that are useful in the unit dosage forms provided herein include antibodies, antibody fragments, anticoagulants, blood factors, bone morphogenetic proteins, enzymes, fusion proteins, growth factors, hormones, interferons, interleukins, and thrombolytics. Further non-limiting examples of biologics that are useful in the unit dosage forms provided herein include any biologic for which there is a biomarker that can be used to determine appropriate subcutaneous dosing of the biologic, e.g., IgG levels can be used to determine subcutaneous dosing of an FcRn antagonist. In an embodiment, the biomarker is present in healthy subjects and/or test animals, such that analysis in healthy volunteers or test animals can be used to determine subcutaneous dosing of the biologic.

In an embodiment, the biologic antagonizes FcRn binding to an antibody Fc region. In an embodiment, the biologic is an antibody, for example, an anti-FcRn antibody. Any anti-FcRn antibody is suitable for use in the unit dosage forms disclosed herein. In an embodiment, the antibody is rozanolixizumab (UCB7665), nipocalimab (M281), orilanolimab (ALXN1830/SYNT001), or batoclimab (IMVT-1401/RVT1401/HBM9161).

In an embodiment, the biologic comprises or consists of a variant Fc region, or FcRn binding fragment thereof, which binds to FcRn with a higher affinity at pH5.5 as compared to a corresponding wild-type Fc region.

In an embodiment, the variant Fc region, or FcRn binding fragment thereof consists of two Fe domains. In an embodiment, the amino acid sequence of the Fe domains of the variant Fc region comprises the amino acid sequence of SEQ ID NO: 1. In an embodiment, the amino acid sequence of the Fe domains of the variant Fc region consists of the amino acid sequence of SEQ ID NO: 1. In an embodiment, the amino acid sequence of the Fe domains of the variant Fc region comprises the amino acid sequence of SEQ ID NO: 2. In an embodiment, the amino acid sequence of the Fe domains of the variant Fc region consists of the amino acid sequence of SEQ ID NO: 2. In an embodiment, the amino acid sequence of the Fe domains of the variant Fc region comprises the amino acid sequence of SEQ ID NO: 3. In an embodiment, the amino acid sequence of the Fe domains of the variant Fc region consists of the amino acid sequence of SEQ ID NO: 3.

In an embodiment, the isolated FcRn antagonist consists of a variant Fc region, wherein the variant Fc region consists of two Fe domains which form a homodimer, wherein the amino acid sequence of each of the Fe domains consists of SEQ ID NO: 1.

In an embodiment, the isolated FcRn antagonist consists of a variant Fc region, wherein the variant Fc region consists of two Fe domains which form a homodimer, wherein the amino acid sequence of each of the Fe domains consists of SEQ ID NO: 2.

In an embodiment, the isolated FcRn antagonist consists of a variant Fc region, wherein the variant Fc region consists of two Fe domains which form a homodimer, wherein the amino acid sequence of each of the Fe domains consists of SEQ ID NO: 3.

In an embodiment, the biologic is efgartigimod (CAS Registry No. 1821402-21-4).

TABLE 1
Amino acid sequences of variant Fc regions

| SEQ ID | |
|---|---|
| NO: | Amino Acid Sequence |
| 1 | CPPCPAPELLGGPSVFLFPPKPKDTLYITREPEVTCVVVDV |
| | SHEDPEVKFNWYVDGVEVHNAKTKPREEQYNSTYRVVSVLT |
| | VLHQDWLNGKEYKCKVSNKALPAPIEKTISKAKGQPREPQV |
| | YTLPPSRDELTKNQVSLTCLVKGFYPSDIAVEWESNGQPEN |
| | NYKTTPPVLDSDGSFFLYSKLTVDKSRWQQGNVFSCSVMHE |
| | ALKFHYTQKSLSLSPG |
| 2 | DKTHTCPPCPAPELLGGPSVFLFPPKPKDTLYITREPEVTC |
| | VVVDVSHEDPEVKFNWYVDGVEVHNAKTKPREEQYNSTYRV |
| | VSVLTVLHQDWLNGKEYKCKVSNKALPAPIEKTISKAKGQP |
| | REPQVYTLPPSRDELTKNQVSLTCLVKGFYPSDIAVEWESN |
| | GQPENNYKTTPPVLDSDGSFFLYSKLTVDKSRWQQGNVFSC |
| | SVMHEALKFHYTQKSLSLSPGK |
| 3 | DKTHTCPPCPAPELLGGPSVFLFPPKPKDTLYITREPEVTC |
| | VVVDVSHEDPEVKFNWYVDGVEVHNAKTKPREEQYNSTYRV |
| | VSVLTVLHQDWLNGKEYKCKVSNKALPAPIEKTISKAKGQP |
| | REPQVYTLPPSRDELTKNQVSLTCLVKGFYPSDIAVEWESN |
| | GQPENNYKTTPPVLDSDGSFFLYSKLTVDKSRWQQGNVFSC |
| | SVMHEALKFHYTQKSLSLSPG |

### Methods of Use

In one aspect, the instant disclosure provides a method of treating a disease or disorder comprising subcutaneously administering a unit dosage form of a biologic disclosed herein, to a subject in need thereof.

In certain embodiments, the instant disclosure provides a method of treating an antibody-mediated autoimmune disease comprising subcutaneously administering a unit dosage form of a variant Fc region disclosed herein, or FcRn binding fragment thereof, to a subject in need thereof.

In an embodiment, the autoimmune disease is selected from the group consisting of allogenic islet graft rejection, alopecia areata, ankylosing spondylitis, antiphospholipid syndrome, autoimmune Addison's disease, Alzheimer's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenic purpura or idiopathic thrombocytopenia purpura or immune mediated thrombocytopenia), autoimmune urticaria, Behcet's disease, bullous pemphigoid (BP), cardiomyopathy, Castleman's syndrome, celiac spruce-dermatitis, chronic fatigue immune disfunction syndrome, chronic inflammatory demyelinating polyneuropathy (CIDP), Churg-Strauss syndrome, cicatricial pemphigoid, CREST syndrome, cold agglutinin disease, Crohn's disease, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic membranous neuropathy, idiopathic pulmonary fibrosis, IgA neuropathy, IgM polyneuropathies, juvenile arthritis, Kawasaki's disease, lichen planus, lichen sclerosus, lupus erythematosus, Meniere's disease, mixed connective tissue disease, mucous membrane pemphigoid, multiple sclerosis, Type 1 diabetes mellitus, multifocal motor neuropathy (MMN), myasthenia gravis (MG), paraneoplastic bullous pemphigoid, pemphigoid gestationis, pemphigus vulgaris (PV), pemphigus foliaceus (PF), pernicious anemia, polyarteritis nodosa, polychrondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Raynaud's phenomenon, Reiter's syndrome, rheumatoid arthritis, sarcoidosis, scleroderma, Sjögren's syndrome, solid organ transplant rejection, stiff-man syndrome, systemic lupus erythematosus, Takayasu's arteritis, toxic epidermal necrolysis (TEN), Stevens-Johnson syndrome (SJS), temporal arteritis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, and Wegner's granulomatosis.

In an embodiment, the variant Fc region, or FcRn binding fragment thereof, is administered once weekly. In an embodiment, the variant Fc region, or FcRn binding fragment thereof, is administered once every two weeks. In an embodiment, the variant Fc region, or FcRn binding fragment thereof, is administered once every 10-14 days. In an embodiment, the variant Fc region, or FcRn binding fragment thereof, is administered once every three weeks. In an embodiment, the variant Fc region, or FcRn binding fragment thereof, is administered once every four weeks.

In an embodiment, the dose of the variant Fc region, or FcRn binding fragment thereof, is about 950 mg, about 975 mg, about 1000 mg, about 1025 mg, or about 1050 mg. In an embodiment, the dose of the variant Fc region, or FcRn binding fragment thereof, is about 950 mg. In an embodiment, the dose of the variant Fc region, or FcRn binding fragment thereof, is about 975 mg. In an embodiment, the dose of the variant Fc region, or FcRn binding fragment thereof, is about 1000 mg. In an embodiment, the dose of the variant Fc region, or FcRn binding fragment thereof, is about 1025 mg. In an embodiment, the dose of the variant Fc region, or FcRn binding fragment thereof, is about 1050 mg.

In an embodiment, the variant Fc region, or FcRn binding fragment thereof, is administered once weekly. In an embodiment, the weekly dose is about 950 mg, about 975 mg, about 1000 mg, about 1025 mg, or about 1050 mg. In an embodiment, the weekly dose is about 950 mg. In an embodiment, the weekly dose is about 975 mg. In an embodiment, the weekly dose is about 1000 mg. In an embodiment, the weekly dose is about 1025 mg. In an embodiment, the weekly dose is about 1050 mg.

In an embodiment, the treatment comprises at least 2 weekly doses. In an embodiment, the treatment comprises at least 3 weekly doses. In an embodiment, the treatment comprises at least 4 weekly doses. In an embodiment, the treatment comprises at least 5 weekly doses. In an embodiment, the treatment comprises at least 6 weekly doses. In an embodiment, the treatment comprises at least 7 weekly doses. In an embodiment, the treatment comprises at least 8 weekly doses. In an embodiment, the treatment comprises at more than 8 weekly doses.

In an embodiment, the dose is an injection. In an embodiment, the dose is a unit dosage form.

In an embodiment, the variant Fc region, or FcRn binding fragment thereof, is administered with a recombinant enzyme human hyaluronidase. In an embodiment, the recombinant enzyme human hyaluronidase is rHuPH20. In an embodiment, the recombinant enzyme human hyaluronidase and the variant Fc region, or FcRn binding fragment thereof, are contained in the same formulation. In an embodiment, the recombinant enzyme human hyaluronidase and the variant Fc region, or FcRn binding fragment thereof, are contained in the separate formulations.

In an embodiment, efgartigimod, is administered with a recombinant enzyme human hyaluronidase. In an embodiment, the recombinant enzyme human hyaluronidase is rHuPH20. In an embodiment, the recombinant enzyme human hyaluronidase and efgartigimod are contained in the same formulation. In an embodiment, the recombinant enzyme human hyaluronidase and efgartigimod are contained in the separate formulations.

In an embodiment, a total serum IgG reduction in the patient of about 60% compared to baseline IgG level is obtained. In an embodiment, a total serum IgG reduction in the patient of about 65%, about 70%, about 75%, or about 80% compared to baseline IgG level is obtained. In an embodiment, a total serum IgG reduction in the patient of about 65% compared to baseline IgG level is obtained. In an embodiment, a total serum IgG reduction in the patient of about 70% compared to baseline IgG level is obtained. In an embodiment, a total serum IgG reduction in the patient of about 75% compared to baseline IgG level is obtained. In an embodiment, a total serum IgG reduction in the patient of about 80% compared to baseline IgG level is obtained.

In an embodiment, the percentage of total serum IgG reduction in the patient is achieved within 1 month from the first dose. In an embodiment, the percentage of total serum IgG reduction in the patient is achieved within 2 weeks, 3 weeks, 4 weeks, 5 weeks, or 6 weeks from the first dose. In an embodiment, the percentage of total serum IgG reduction in the patient is achieved within 2 weeks from the first dose. In an embodiment, the percentage of total serum IgG reduction in the patient is achieved within 3 weeks from the first dose. In an embodiment, the percentage of total serum IgG reduction in the patient is achieved within 4 weeks from the first dose. In an embodiment, the percentage of total serum IgG reduction in the patient is achieved within 5 weeks from the first dose. In an embodiment, the percentage of total serum IgG reduction in the patient is achieved within 6 weeks from the first dose. In an embodiment, the percentage of total serum IgG reduction in the patient is achieved within 31, 30, 29, 28, 27, 26, or 25 days from the first dose.

In an embodiment, the maximum percentage of total serum IgG reduction in the patient is achieved within 1 month from the first dose. In an embodiment, the maximum percentage of total serum IgG reduction in the patient is achieved within 2 weeks, 3 weeks, 4 weeks, 5 weeks, or 6 weeks from the first dose. In an embodiment, the maximum percentage of total serum IgG reduction in the patient is achieved within 2 weeks from the first dose. In an embodiment, the maximum percentage of total serum IgG reduction in the patient is achieved within 3 weeks from the first dose. In an embodiment, the maximum percentage of total serum IgG reduction in the patient is achieved within 4 weeks from the first dose. In an embodiment, the maximum percentage of total serum IgG reduction in the patient is achieved within 5 weeks from the first dose. In an embodiment, the maximum percentage of total serum IgG reduction in the patient is achieved within 6 weeks from the first dose. In an embodiment, the maximum percentage of total serum IgG reduction in the patient is achieved within 31, 30, 29, 28, 27, 26, or 25 days from the first dose.

In an embodiment, the total serum IgG level in the patient is reduced to 2000 to 4000 μg/mL. In an embodiment, the total serum IgG level in the patient is reduced to 2000 to 3000 μg/mL. In an embodiment, the total serum IgG level in the patient is reduced to 3000 to 4000 μg/mL. In an embodiment, the total serum IgG level in the patient is reduced to 2500 to 3500 μg/mL. In an embodiment, the total serum IgG level in the patient is reduced to 2750 to 3250 μg/mL.

In an embodiment, the total serum IgG in the patient is analyzed using a bioanalytical method. In an embodiment, the total serum IgG in the patient is analyzed using ELISA or automated diagnostic analyzer (IVD). In an embodiment, the total serum IgG in the patient is analyzed using ELISA. In an embodiment, the total serum IgG in the patient is analyzed using automated diagnostic analyzer (IVD).

In an embodiment, at least one of the IgG subtypes is reduced. In an embodiment, IgG1 is reduced. In an embodiment, IgG2 is reduced. In an embodiment, IgG3 is reduced. In an embodiment, IgG4 is reduced.

In an embodiment, the variant Fc region is efgartigimod.

In one aspect, provided herein is a variant Fc region, or FcRn binding fragment thereof, wherein the Fc domains of the Fc region comprise the amino acids Y, T, E, K, F, and Y at EU Kabat positions 252, 254, 256, 433, 434, and 436 respectively, for use in treating myasthenia gravis in a human patient.

In one aspect, the instant disclosure provides a variant Fc region, or FcRn binding fragment thereof, wherein the Fc domains of the Fc region comprise the amino acids Y, T, E, K, F, and Y at EU Kabat positions 252, 254, 256, 433, 434, and 436 respectively, for use in treating myasthenia gravis in a human patient, wherein: the variant Fc region, or FcRn binding fragment thereof, is administered subcutaneously at a weekly dose of between 950 and 1050 mg, independent of the weight of the patient, and a total serum IgG reduction in the patient of at least 60% compared to baseline IgG level is obtained.

In an embodiment, the weekly dose is about 950 mg, about 975 mg, about 1000 mg, about 1025 mg, or about 1050 mg. In an embodiment, the weekly dose is about 950 mg. In an embodiment, the weekly dose is about 975 mg. In an embodiment, the weekly dose is about 1000 mg. In an embodiment, the weekly dose is about 1025 mg. In an embodiment, the weekly dose is about 1050 mg.

In an embodiment, the treatment comprises at least 2 weekly doses. In an embodiment, the treatment comprises at least 3 weekly doses. In an embodiment, the treatment comprises at least 4 weekly doses. In an embodiment, the treatment comprises at least 5 weekly doses. In an embodiment, the treatment comprises at least 6 weekly doses. In an embodiment, the treatment comprises at least 7 weekly doses. In an embodiment, the treatment comprises at least 8 weekly doses. In an embodiment, the treatment comprises at more than 8 weekly doses.

In an embodiment, the dose is an injection. In an embodiment, the dose is a unit dosage form.

In an embodiment, the variant Fc region, or FcRn binding fragment thereof, is administered with a recombinant enzyme human hyaluronidase. In an embodiment, the recombinant enzyme human hyaluronidase is rHuPH20. In an embodiment, the recombinant enzyme human hyaluronidase and the variant Fc region, or FcRn binding fragment thereof, are contained in the same formulation. In an embodiment, the recombinant enzyme human hyaluronidase and the variant Fc region, or FcRn binding fragment thereof, are contained in the separate formulations. In an embodiment, the recombinant enzyme human hyaluronidase and the variant Fc region, or FcRn binding fragment thereof, are co-administered. In an embodiment, the recombinant enzyme human hyaluronidase and the variant Fc region, or FcRn binding fragment thereof, are administered sequentially. In an embodiment, the recombinant enzyme human hyaluronidase is administered before the variant Fc region, or FcRn binding fragment thereof. In an embodiment, the recombinant enzyme human hyaluronidase is administered after the variant Fc region, or FcRn binding fragment thereof.

In an embodiment, efgartigimod, is administered with a recombinant enzyme human hyaluronidase. In an embodiment, the recombinant enzyme human hyaluronidase is rHuPH20. In an embodiment, the recombinant enzyme human hyaluronidase and efgartigimod are contained in the same formulation. In an embodiment, the recombinant enzyme human hyaluronidase and efgartigimod are contained in the separate formulations. In an embodiment, the recombinant enzyme human hyaluronidase and efgartigimod are co-administered. In an embodiment, the recombinant enzyme human hyaluronidase and efgartigimod are administered sequentially. In an embodiment, the recombinant enzyme human hyaluronidase is administered before efgartigimod. In an embodiment, the recombinant enzyme human hyaluronidase is administered after efgartigimod.

In an embodiment, a total serum IgG reduction in the patient of about 60% compared to baseline IgG level is obtained. In an embodiment, a total serum IgG reduction in the patient of about 65%, about 70%, about 75%, or about 80% compared to baseline IgG level is obtained. In an embodiment, a total serum IgG reduction in the patient of about 65% compared to baseline IgG level is obtained. In an embodiment, a total serum IgG reduction in the patient of about 70% compared to baseline IgG level is obtained. In an embodiment, a total serum IgG reduction in the patient of about 75% compared to baseline IgG level is obtained. In an embodiment, a total serum IgG reduction in the patient of about 80% compared to baseline IgG level is obtained.

In an embodiment, the percentage of total serum IgG reduction in the patient is achieved within 1 month from the first dose. In an embodiment, the percentage of total serum IgG reduction in the patient is achieved within 2 weeks, 3 weeks, 4 weeks, 5 weeks, or 6 weeks from the first dose. In an embodiment, the percentage of total serum IgG reduction in the patient is achieved within 2 weeks from the first dose. In an embodiment, the percentage of total serum IgG reduction in the patient is achieved within 3 weeks from the first dose. In an embodiment, the percentage of total serum IgG reduction in the patient is achieved within 4 weeks from the first dose. In an embodiment, the percentage of total serum IgG reduction in the patient is achieved within 5 weeks from the first dose. In an embodiment, the percentage of total serum IgG reduction in the patient is achieved within 6 weeks from the first dose. In an embodiment, the percentage of total serum IgG reduction in the patient is achieved within 31, 30, 29, 28, 27, 26, or 25 days from the first dose.

In an embodiment, the maximum percentage of total serum IgG reduction in the patient is achieved within 1 month from the first dose. In an embodiment, the maximum percentage of total serum IgG reduction in the patient is achieved within 2 weeks, 3 weeks, 4 weeks, 5 weeks, or 6 weeks from the first dose. In an embodiment, the maximum percentage of total serum IgG reduction in the patient is achieved within 2 weeks from the first dose. In an embodiment, the maximum percentage of total serum IgG reduction in the patient is achieved within 3 weeks from the first dose. In an embodiment, the maximum percentage of total serum IgG reduction in the patient is achieved within 4 weeks from the first dose. In an embodiment, the maximum percentage of total serum IgG reduction in the patient is achieved within 5 weeks from the first dose. In an embodiment, the maximum percentage of total serum IgG reduction in the patient is achieved within 6 weeks from the first dose. In an embodiment, the maximum percentage of total serum IgG reduction in the patient is achieved within 31, 30, 29, 28, 27, 26, or 25 days from the first dose.

In an embodiment, the total serum IgG level in the patient is reduced to 2000 to 4000 μg/mL. In an embodiment, the total serum IgG level in the patient is reduced to 2000 to 3000 μg/mL. In an embodiment, the total serum IgG level in the patient is reduced to 3000 to 4000 μg/mL. In an embodiment, the total serum IgG level in the patient is reduced to 2500 to 3500 μg/mL. In an embodiment, the total serum IgG level in the patient is reduced to 2750 to 3250 μg/mL.

In an embodiment, the total serum IgG in the patient is analyzed using a bioanalytical method. In an embodiment, the total serum IgG in the patient is analyzed using ELISA or automated diagnostic analyzer (IVD). In an embodiment, the total serum IgG in the patient is analyzed using ELISA. In an embodiment, the total serum IgG in the patient is analyzed using automated diagnostic analyzer (IVD).

In an embodiment, at least one of the IgG subtypes is reduced. In an embodiment, IgG1 is reduced. In an embodiment, IgG2 is reduced. In an embodiment, IgG3 is reduced. In an embodiment, IgG4 is reduced.

In an embodiment, the variant Fc region is efgartigimod.

Also provided herein is a variant Fc region, or FcRn binding fragment thereof, wherein the Fe domains of the Fc region comprise the amino acids Y, T, E, K, F, and Y at EU Kabat positions 252, 254, 256, 433, 434, and 436 respectively, for use in treating pemphigus vulgaris in a human patient.

In one aspect, the instant disclosure provides a variant Fc region, or FcRn binding fragment thereof, wherein the Fe domains of the Fc region comprise the amino acids Y, T, E, K, F, and Y at EU Kabat positions 252, 254, 256, 433, 434, and 436 respectively, for use in treating pemphigus vulgaris in a human patient, wherein: the variant Fc region, or FcRn binding fragment thereof, is administered subcutaneously at a weekly dose of between 1950 and 2050 mg, independent of the weight of the patient, and a total serum IgG reduction in the patient of at least 60% compared to baseline IgG level is obtained.

In an embodiment, the weekly dose is about 1950 mg, about 1975 mg, about 2000 mg, about 2025 mg, or about 2050 mg. In an embodiment, the weekly dose is about 1950 mg. In an embodiment, the weekly dose is about 1975 mg. In an embodiment, the weekly dose is about 2000 mg. In an embodiment, the weekly dose is about 2025 mg. In an embodiment, the weekly dose is about 2050 mg.

In an embodiment, the treatment comprises at least 2 weekly doses. In an embodiment, the treatment comprises at least 3 weekly doses. In an embodiment, the treatment comprises at least 4 weekly doses. In an embodiment, the treatment comprises at least 5 weekly doses. In an embodiment, the treatment comprises at least 6 weekly doses. In an embodiment, the treatment comprises at least 7 weekly doses. In an embodiment, the treatment comprises at least 8 weekly doses. In an embodiment, the treatment comprises at more than 8 weekly doses.

In an embodiment, the dose is a unit dosage form.

In an embodiment, the variant Fc region, or FcRn binding fragment thereof, is administered with a recombinant enzyme human hyaluronidase. In an embodiment, the recombinant enzyme human hyaluronidase is rHuPH20. In an embodiment, the recombinant enzyme human hyaluronidase and the variant Fc region, or FcRn binding fragment thereof, are contained in the same formulation. In an embodiment, the recombinant enzyme human hyaluronidase and the variant Fc region, or FcRn binding fragment thereof, are contained in the separate formulations.

In an embodiment, efgartigimod, is administered with a recombinant enzyme human hyaluronidase. In an embodiment, the recombinant enzyme human hyaluronidase is rHuPH20. In an embodiment, the recombinant enzyme human hyaluronidase and efgartigimod are contained in the same formulation. In an embodiment, the recombinant enzyme human hyaluronidase and efgartigimod are contained in the separate formulations.

In an embodiment, a total serum IgG reduction in the patient of about 60% compared to baseline IgG level is obtained. In an embodiment, a total serum IgG reduction in the patient of about 65%, about 70%, about 75%, or about 80% compared to baseline IgG level is obtained. In an embodiment, a total serum IgG reduction in the patient of about 65% compared to baseline IgG level is obtained. In an embodiment, a total serum IgG reduction in the patient of about 70% compared to baseline IgG level is obtained. In an embodiment, a total serum IgG reduction in the patient of about 75% compared to baseline IgG level is obtained. In an embodiment, a total serum IgG reduction in the patient of about 80% compared to baseline IgG level is obtained.

In an embodiment, the percentage of total serum IgG reduction in the patient is achieved within 1 month from the first dose. In an embodiment, the percentage of total serum IgG reduction in the patient is achieved within 2 weeks, 3 weeks, 4 weeks, 5 weeks, or 6 weeks from the first dose. In an embodiment, the percentage of total serum IgG reduction in the patient is achieved within 2 weeks from the first dose. In an embodiment, the percentage of total serum IgG reduction in the patient is achieved within 3 weeks from the first dose. In an embodiment, the percentage of total serum IgG reduction in the patient is achieved within 4 weeks from the first dose. In an embodiment, the percentage of total serum IgG reduction in the patient is achieved within 5 weeks from the first dose. In an embodiment, the percentage of total serum IgG reduction in the patient is achieved within 6 weeks from the first dose.

In an embodiment, the maximum percentage of total serum IgG reduction in the patient is achieved within 1 month from the first dose. In an embodiment, the maximum percentage of total serum IgG reduction in the patient is achieved within 2 weeks, 3 weeks, 4 weeks, 5 weeks, or 6 weeks from the first dose. In an embodiment, the maximum percentage of total serum IgG reduction in the patient is achieved within 2 weeks from the first dose. In an embodiment, the maximum percentage of total serum IgG reduction in the patient is achieved within 3 weeks from the first dose. In an embodiment, the maximum percentage of total serum IgG reduction in the patient is achieved within 4 weeks from the first dose. In an embodiment, the maximum percentage of total serum IgG reduction in the patient is achieved within 5 weeks from the first dose. In an embodiment, the maximum percentage of total serum IgG reduction in the patient is achieved within 6 weeks from the first dose.

In an embodiment, the total serum IgG level in the patient is reduced to 2000 to 4000 μg/mL. In an embodiment, the total serum IgG level in the patient is reduced to 2000 to 3000 μg/mL. In an embodiment, the total serum IgG level in the patient is reduced to 3000 to 4000 μg/mL. In an embodiment, the total serum IgG level in the patient is reduced to 2500 to 3500 μg/mL. In an embodiment, the total serum IgG level in the patient is reduced to 2750 to 3250 μg/mL.

In an embodiment, the total serum IgG in the patient is analyzed using a bioanalytical method. In an embodiment, the total serum IgG in the patient is analyzed using ELISA or automated diagnostic analyzer (IVD). In an embodiment, the total serum IgG in the patient is analyzed using ELISA. In an embodiment, the total serum IgG in the patient is analyzed using automated diagnostic analyzer (IVD).

In an embodiment, at least one of the IgG subtypes is reduced. In an embodiment, IgG1 is reduced. In an embodiment, IgG2 is reduced. In an embodiment, IgG3 is reduced. In an embodiment, IgG4 is reduced.

In an embodiment, the variant Fc region is efgartigimod.

### EXAMPLES

The following examples are offered by way of illustration, and not by way of limitation.

### Example 1: Study Comparing the PK/PD and Safety of Subcutaneous Doses of Efgartigimod+rHuPH20

Efgartigimod (UNII: 961YV20515) is a human lgG1-derived Fc fragment of the za allotype (a variant Fc region) that binds with nanomolar affinity to human FcRn. A randomized, open-label, clinical trial was performed to evaluate the safety and pharmacokinetic (PK)/pharmacodynamic (PD) parameters of subcutaneous (SC) doses of efgartigimod.

An SC formulation with recombinant human hyaluronidase PH20 enzyme (rHuPH20) has been developed for SC administration of efgartigimod as an alternative to IV infusion. The enzyme rHuPH20 locally degrades hyaluronan (HA) in the SC space, which allows for increased dispersion and absorption of co-administered therapies. The ready-to-use liquid SC formulation comprising efgartigimod and rHuPH20 (efgartigimod-PH20) was injected as a fixed dose. This formulation and method of administration are expected to increase patient convenience compared to the IV formulation and administration.

Healthy volunteers aged 18-70 years, with body weight in the range of 50-100 kg were screened for 21 days and then randomized into four treatment groups (n=8 for each group), as follows:
a. Treatment A: single SC dose of 750 mg efgartigimod co-administered with 2000 U/mL the hyaluronidase enzyme, rHuPH20;
b. Treatment B: single SC dose of 1250 mg efgartigimod co-administered with 2000 U/mL rHuPH20;
c. Treatment C: single SC dose of 1750 mg efgartigimod co-administered with 2000 U/mL rHuPH20; and
d. Treatment D: single SC dose of 10 mg/kg efgartigimod co-administered with 2000 U/mL rHuPH20.

### Analysis of Pharmacokinetic Parameters

An interim analysis of several pharmacokinetic parameters was performed based on the PK population (randomized patients who had at least one plasma concentration value available for efgartigimod). Plasma concentrations of efgartigimod at each sampling time point were analyzed by the following summary statistics: arithmetic mean calculated using untransformed data, standard deviation (SD) calculated using untransformed data, minimum, median, maximum, number of observations, and number of observations > lower limit of quantification (LLOQ).

Geometric mean plasma concentrations against protocol time were shown by patient in both linear and log scales, respectively.

The following summary statistics were assessed for all the PK parameters except for tₘₐₓ: Gₘₑₐₙ, GCV, arithmetic mean calculated using untransformed data, SD calculated using untransformed data, minimum, median, maximum, and number of observations.

The following summary statistics were assessed for the PK parameters tₘₐₓ: number of observations, median, minimum, and maximum.

### Analysis of Pharmacodynamic Parameters

Continuous PD parameters, including analysis of total IgG were summarized with descriptive statistics including geometric mean.

### Results

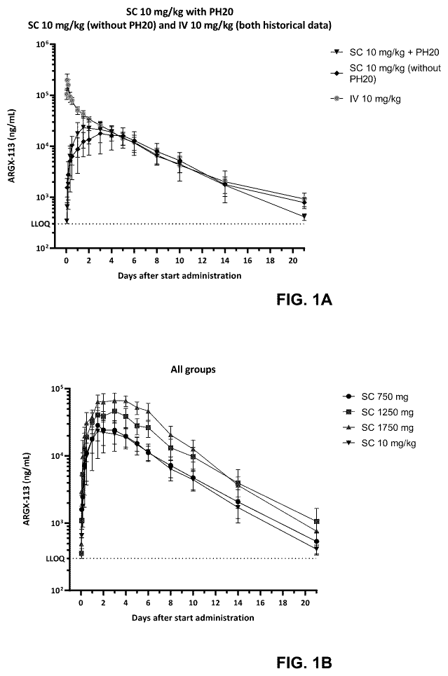

An interim analysis was performed 22 days after the doses were administered to evaluate PK and PD parameters. Serum levels of efgartigimod following the single SC doses in patients in treatment groups A-D were compared to historical data from administration of 10 mg/kg IV or SC efgartigimod (without rHuPH20) (FIG. 1A and FIG. 1B). The PK data shows that the addition of rHuPH20 resulted in increased bioavailability of efgartigimod following SC administration compared to SC administration without rHuPH20 (see Table 2).

TABLE 2
PK parameters from interim analysis

| | With PH20 (1901 data) | | | | | | | | without PH20 (1702 data) | | | |
|---|---|---|---|---|---|---|---|---|---|---|---|---|
| | 750 mg SC | | 1250 mg SC | | 1750 mg SC | | 10 mg/kg SC | | 10 mg/kg SC | | 10 mg/kg IV | |
| | Average | sd | Average | sd | Average | sd | Average | sd | Average | Sd | Average | sd |
| Cₘₐₓ | 30850 | 10305 | 51388 | 10672 | 78438 | 10209 | 25600 | 12886 | 19435 | | 205750 | |
| (ng/mL) | | | | | | | | | | | | |
| Tₘₐₓ | 1.88 | 1.09 | 1.94 | 0.90 | 3.56 | 1.29 | 2.44 | 0.94 | 3.00 | | | |
| AUC | 164594 | 13399 | 308672 | 23050 | 469629 | 31367 | 151130 | 14017 | 137530 | 15588 | 265815 | 13188 |
| (days | | | | | | | | | | | | |
| ng/mL) | | | | | | | | | | | | |

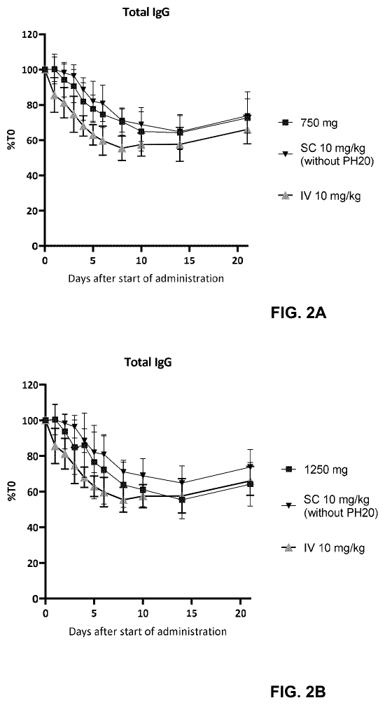

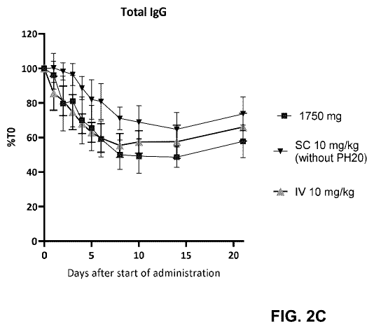

PD results from the interim analysis were also compared with historical data. The total IgG reduction following 750 mg SC efgartigimod was inferior to 10 mg/kg IV administration (FIG. 2A), while the maximum IgG reduction following 1250 mg SC efgartigimod was comparable to 10 mg/kg IV administration (FIG. 2B). Both the onset of total IgG reduction and the prolonged effect of total IgG reduction following 1750 mg SC efgartigimod were comparable to 10 mg/kg IV administration (FIG. 2C). No significant adverse events were observed in treatment groups A-D.

This single dose trial demonstrated the safety of SC administration of efgartigimod, co-administered with rHuPH20, and indicated that SC administration could result in a total IgG reduction that is comparable to IV administration in healthy volunteers.

### Example 2: Calculation of Subcutaneous Dose of Efgartigimod from Pharmacokinetic (PK) and Pharmacodynamic (PD) Data

To determine a safe and effective SC dose of a biologic, PK/PD modeling was used to match reduction of total IgG (a PD parameter) of an IV and SC dose of a biologic, based on data from single SC administrations of the biologic, using a known IV dose as a benchmark.

A previously determined PK/PD model was used to construct simulations of total IgG reduction following different subcutaneous doses of efgartigimod, with and without the hyaluronidase enzyme rHuPH20. Using preliminary PK/PD data obtained from human subjects who were treated with single subcutaneous doses of efgartigimod (the study described in Example 1 above), the PK/PD model was used to describe the Cₘₐₓ and the AUC, with or without rHuPH20, and the median trend of IgG reduction across dose groups.

A covariate analysis for body weight showed that there is no statistically significant effect of body weight on PK or IgG, suggesting that a fixed dose is possible for subcutaneous administration.

### Previous PK Model for EFgartigimod in Healthy Volunteers

Previously, a population PK analysis was performed to assess the effects of efgartigimod in a study of efgartigimod in healthy volunteers. This was a Phase I, randomized, double-blind, placebo-controlled, single and multiple ascending IV dose study to assess the safety, tolerability, PK, PD, and immunogenicity of efgartigimod in healthy male and female volunteers of non-child bearing potential. In summary, the PK model adequately captured the concentration-time profiles for efgartigimod, after single ascending doses of 0.2, 2, 10, 25, and 50 mg/kg and multiple ascending doses. Multiple doses of efgartigimod or placebo were given every 4 days (q4d) on 6 occasions (10 mg/kg alone) or every 7 days (q7d) on 4 occasions (10 and 25 mg/kg). The final PK model consisted of a three-compartmental model with linear clearance and it included the assumption that the second peripheral volume (V3) was equal to the first peripheral volume (V2). Inter-individual variability (IIV) was identified for clearance (CL), the central volume of distribution (V1), the inter-compartmental clearance (Q), and the volume of the peripheral compartments (V2=V3). Furthermore, covariance for the IIV was implemented in the model for CL, V1, and V2=V3. An additive residual error model was used, which is standard for log-transformed data.

This model was extended to describe the PK of efgartigimod in healthy volunteers in another efgartigimod study. This was a randomized, open-label, parallel group study to compare the PK, PD, safety, and tolerability of SC formulation with intravenous (IV) formulation of efgartigimod in healthy male subjects. In this trial, the subjects were assigned to either treatment A (single-dose of 10 mg/kg IV) or B (single-dose of 10 mg/kg SC) or to treatment C (two IV doses of 20 mg/kg, followed by 8 weekly SC doses of 300 mg). To describe the PK of the compound in this study, a zero-order absorption was added to the existing PK model and duration of the zero-order process (DUR) as well as the absolute bioavailability (F) were estimated. The final model included IIV on CL, V2=V3, V1, Q2, and F. To increase model stability, only covariance between IIV on CL and V2=V3 was estimated.

Updated Modeling Approach and Assumptions for Efgartigimod Co-Administered with rHuPH20

The focus of the analysis was the modelling of data from 32 subjects treated with a single SC injection of either 750 mg, 1250 mg, 1750 mg, or 10 mg/kg of efgartigimod+rHuPH20 (the study described in Example 1). For data on IV and SC dosing without rHuPH20, PK and IgG historical data from treatments A (10 mg/kg single IV dose) and B (10 mg/kg single SC dose) from a previous study were included in the analysis.

First, the parameters from the existing PK model for healthy volunteers were used to predict healthy volunteer data in the study described in Example 1. The model did not adequately predict the PK of efgartigimod co-administered with rHuPH20, especially in the absorption phase. Therefore, the absorption-related parameters (i.e., absolute bioavailability and duration of the zero-order absorption process) were estimated for the study described in Example 1, together with the residual error. In this way, the description of the PK of efgartigimod in the new study improved. However, the absorption phase was not adequately described, yet. To improve the description of the absorption of the compound when co-administered with rHuPH20, the first-order absorption rate constant kA was also estimated (i.e., 0.24 l/h in Table 3) for the study described in Example 1, whereas, in the previous PK model, the parameter kA was fixed to 99, to resemble the zero-order absorption. In this way, a sequential zero-first-order absorption model could be identified and it improved the description of the PK of efgartigimod+rHuPH20. Further, the duration of the zero-order process was estimated to be lower in the study described in Example 1, as compared to the historical data (i.e., 83.7 h vs 131 h, as reported in Table 3).

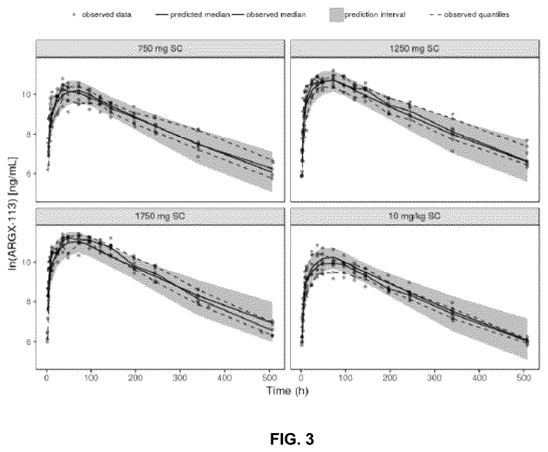

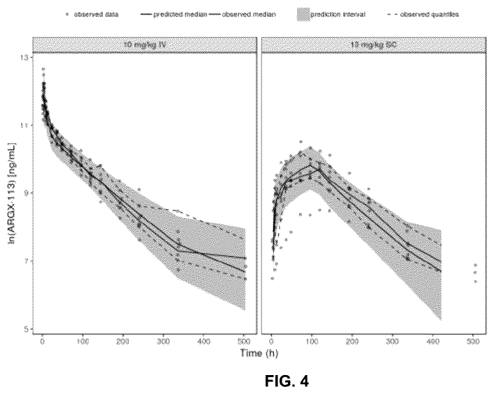

As a last step, all PK parameters were optimized on data from the historical data and the study described in Example 1. Parameter estimates showed that the relative bioavailability and the duration of the zero-order process were found to be higher and lower respectively in the study described in Example 1, as compared to the historical data (see Table 3). Furthermore, inter-individual variability (IIV) on Q2 and the correlation between IIV on clearance (CL) and IIV on the first peripheral volume (V2) were removed, as they were not precisely estimated (i.e., RSE %>50%). To improve model stability, the inter-individual variability on kA was removed and it was estimated on the duration of the zero-order absorption. As shown in the visual predictive checks, the PK model adequately captured the typical profile of efgartigimod concentration as well as inter-individual variability across treatment groups in the study described in Example 1 (see FIG. 3) and in the historical data (see FIG. 4). The effect of body weight was investigated on PK parameters, but it was not found to be statistically significant.

TABLE 3
Parameter estimates from efgartigimod PK model for healthy volunteers

| | Estimate absorption parameters for 1901 | | Final PK (historical data and single SC dosing data) | |
|---|---|---|---|---|
| | | RSE | | RSE |
| parameter (unit) | value | (%)^{a} | Value | (%)^{a} |

Structural parameters

| CL (L/h) | 0.145 | FIX^{d} | — | 0.128 | | 2.5 |
|---|---|---|---|---|---|---|
| V1 (L) | 4.50 | FIX^{d} | — | 3.41 | | 10.2 |
| Q2 (L/h) | 0.00616 | FIX^{d} | — | 0.00612 | | 6.9 |
| V2 = V3 (L) | 7.10 | FIX^{d} | — | 6.74 | | 4.4 |
| Q3 (L/h) | 0.194 | FIX^{d} | — | 0.294 | | 5.9 |
| F(−) SC 1702 | 0.544 | FIX^{d} | — | 0.56 | | 3.3 |
| DUR(h) SC 1702 | 131 | FIX^{d} | — | 114 | | 2.6 |
| F(−) SC 1901 | 0.805 | | 5.2 | 0.764 | | 3.7 |
| DUR(h) SC 1901 | 83.7 | | 1.96 | 76 | | 5.1 |
| k_{A} (1/h) 1702 | 99 | FIX^{d} | — | 99 | FIX^{d} | |

| | | | | |
|---|---|---|---|---|
| k_{A} (1/h) 1901 | 0.24 | 16.1 | 0.155 | 17.7 |

Inter-individual variability

| ω²C1 | 0.0342 | [18.7% ^{b}] FIX^{d} | — | 0.0126 | [11.3% ^{b}] | 50.6 |
|---|---|---|---|---|---|---|
| ω²V1 | 0.102 | [32.8% ^{b}] FIX^{d} | — | 0.554 | [86.0% ^{b}] | 26.1 |
| ω²V2 = V3 | 0.046 | [21.7% ^{b}] FIX^{d} | — | 0.0758 | [28.1% ^{b}] | 38.4 |
| ω²Q2 | 0.274 | [56.1% ^{b}] FIX^{d} | — | — | | — |
| ω²F | 0.164 | [42.2% ^{b}] FIX^{d} | — | 0.776 | [108% ^{b}] | 27.1 |

| | | | | | |
|---|---|---|---|---|---|
| ω²DUR 1901 | — | — | 0.0716 | [27.2% ^{b}] | 29.4 |

| | | | | | |
|---|---|---|---|---|---|
| ω² kA 1901 | 0.638 | [94.5% ^{b}] | 25.9 | — | — |
| ω²CLxV2 = V3 | 0.0266 | [0.656% ^{c}] FIX^{d} | — | — | — |

Residual variability

| | | | | | | |
|---|---|---|---|---|---|---|
| σ²add | 0.0637 | [25.6% ^{b}] | 11.2 | 0.0555 | [23.9% ^{b}] | 12.6 |

^{a}Relative standard error: CV % = 100 * standard error/Value,
^{b} 100 · √e^{ω2} − 1,
^{c} ω_{x, y}/(CV %(x) · CV %(y)),
^{d}value fixed to the estimate from the combined analysis for studies efgartigimod-1501 and efgartigimod-1901
1702 = previous studies (historical data)
1901 = the study described in Example 1

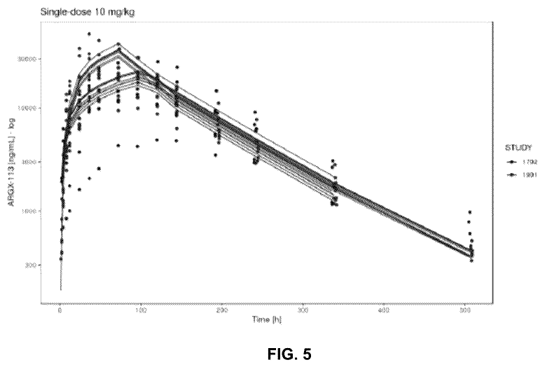

A comparison between 10 mg/kg SC of efgartigimod with and without co-administration of rHuPH20 suggested that the absorption model may still be improved for the study described in Example 1, as the observed tₘₐₓ appeared to be smaller than the predicted tₘₐₓ (FIG. 5). Different absorption models were investigated to improve the description of the PK of efgartigimod in the study described in Example 1, such as the parallel zero-zero-order absorption (with and without lag time) and the parallel zero-first-order absorption (with and without lag time). However, none of these investigated models turned out to be better than the current one with sequential zero-first-order absorption. Therefore, potential dependencies between PK parameters and the dose were investigated. It appeared that the bioavailability increased with dose in the study described in Example 1. Nevertheless, the inclusion of a dose function for the relative bioavailability did not significantly improve the description of the population and individual PK profiles.

In conclusion, the population PK model for efgartigimod+rHuPH20 was deemed adequate for the PK/PD analysis.

### PK Total IgG Model

The PK/total IgG model consisted of an indirect response model in which the concentration of efgartigimod stimulated the degradation rate of total IgG (kₒᵤₜ). This model reflects the mechanism of action for efgartigimod, which binds the FcRn receptor and reduces recycling of total IgG and causes increased degradation of total IgG. An Eₘₐₓ model was used to quantify the PK/PD relationship (with the Eₘₐₓ parameter fixed to the estimate from the combined analysis of previous studies) because the total IgG reduction effect of efgartigimod was found to be saturable. An effect compartment was included in the model to allow for an accurate description of the delay in decrease in total IgG concentrations. Inter-individual variability (IIV) was identified for baseline total IgG levels and for the potency (EC₅₀) of efgartigimod assuming a log-normal distribution and residual variability was described by a proportional error model.

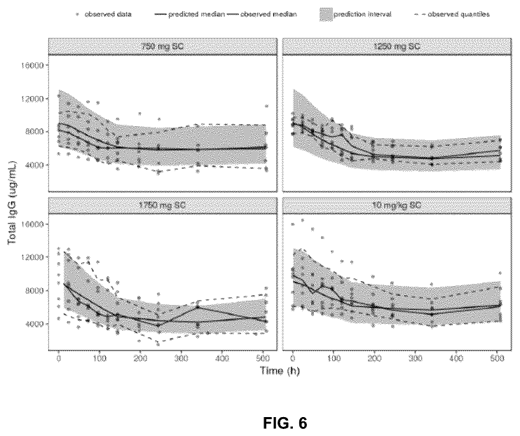

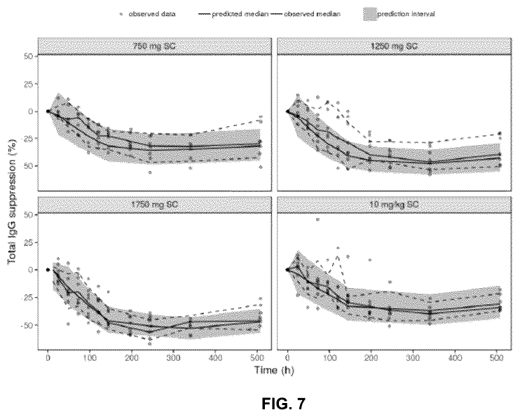

In particular, the model parameters derived from a previous combined analysis for previous efgartigimod studies were used to predict the total IgG concentration in the study described in Example 1. To do so, it was assumed that the baseline of total IgG in the study described in Example 1 was the same as the baseline in one of the previous studies (i.e., 8570 mg/L). Overall, the model could predict 750 mg, 1750 mg, and 10 mg/kg dose groups reasonably well. However, the 1250 mg treatment group was not adequately predicted. By estimating the baseline of total IgG in the study described in Example 1, the model improved the description of total IgG across dose groups (parameter estimates are reported in Table 4). However, it still under-predicted the total IgG concentration in the 1250 mg group. As a further step, all parameters, except Eₘₐₓ, were optimized on total IgG data from a previous study and the study described in Example 1. With respect to the other treatment groups in the study described in Example 1, the inter-individual variability on the baseline in the 1250 mg SC group appeared to be lower. Visual predictive checks showed that the model over-predicted the inter-individual variability in the 1250 mg SC treatment group (FIG. 6). Further, this model under-predicted the median total IgG reduction in the 750 mg and 1750 mg SC dose groups (FIG. 7).

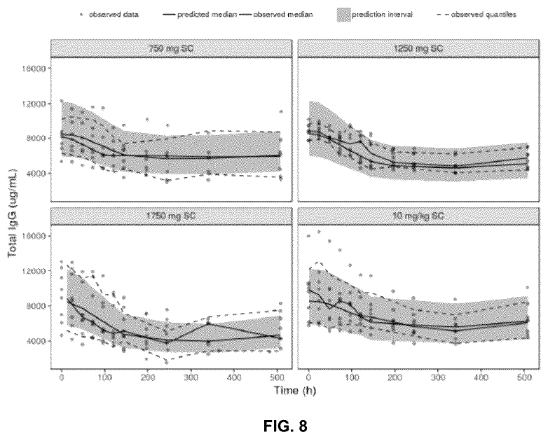

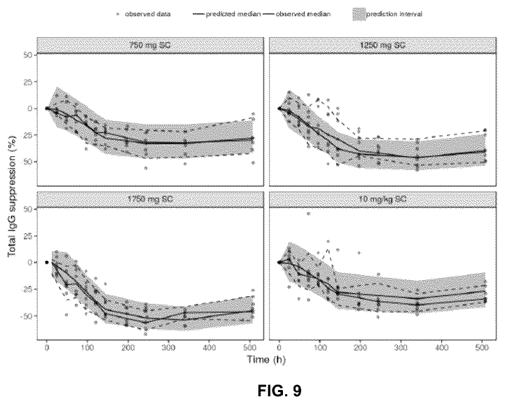

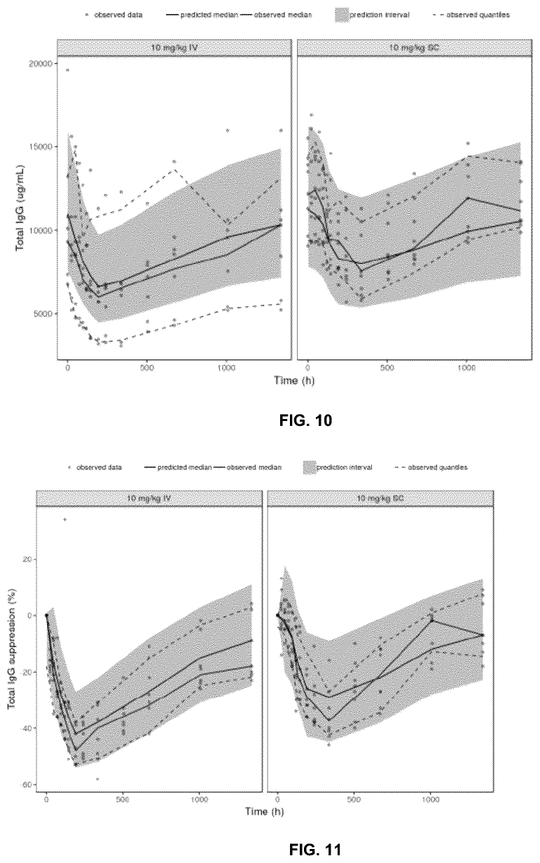

The inclusion of an effect compartment in the model structure allowed better capture of the total IgG concentration in the SC dose groups in both the historical data and the study described in Example 1. With this new model structure, EC₅₀ was estimated to be higher, because it represented the concentration in the effect compartment (i.e., 33636 ng/mL vs 20900 ng/mL in Table 4). The visual predictive checks confirmed that the model captured both the typical total IgG concentrations (FIG. 8) and reduction (FIG. 9) over time and the inter-individual variability in the study described in Example 1. Moreover, the inclusion of the effect compartment provided a reasonable description of the total IgG concentration (FIG. 10) and reduction (FIG. 11) in the historical data. Therefore, this model is considered suitable to explore the expected total IgG reduction in future trials.

The effect of body weight was investigated on the baseline total IgG and EC₅₀ parameters, but it did not turn out to be statistically significant. In conclusion, the population PK/total IgG model for efgartigimod+rHuPH20 was deemed adequate for simulating the typical PK and IgG reduction and its uncertainty to assess the dose in a future trial.

TABLE 4
Parameter estimates from efgartigimod PK/PD model in healthy volunteers

| | Only baseline estimated for the single SC dosing data | | Final PK/PD (historical data and single SC dosing data) | |
|---|---|---|---|---|
| Parameter (unit) | value | RSE(%)^{a} | Value | RSE(%)^{a} |

Structural parameters

| Baseline (mg/L) - 1901 | 9266 | | 4.6 | 8754 | | 4.7 |
|---|---|---|---|---|---|---|
| Baseline (mg/L) - 1702 | 11900 | | — | 11435 | | 7.3 |
| kₒᵤₜ | 0.00179 | FIX^{c} | — | 0.00175 | | 12.6 |
| Eₘₐₓ | 4.7 | FIX^{c} | — | 4.7 | FIX^{c} | — |
| EC₅₀ | 20900 | FIX^{c} | — | 33636 | | 17.6 |
| kₑₒ | — | | — | 0.0288 | | 28.2 |
| Hill coefficient, n | 1 | FIX^{c} | — | 1 | FIX^{c} | — |

Inter-individual variability

| | | | | | | |
|---|---|---|---|---|---|---|
| ω²baseline | 0.0991 | [32.3%^{b}] FIX^{c} | — | 0.0704 | [27.0%^{b}] | 20.5 |
| ω²EC50 | 0.296 | [58.7%^{b}] FIX^{c} | — | 0.111 | [34.3%^{b}] | 44.6 |

Residual variability

| | | | | | | |
|---|---|---|---|---|---|---|
| σ¹² prop | 0.0126 | [11.3%^{b}] | 23.2 | 0.0109 | [10.5%^{b}] | 25.2 |

^{a}Relative standard error: CV % = 100 * standard error/Value,
^{b}100 · √e^{ω2} − 1,
^{c}value fixed to the estimate from the combined analysis for studies the historical data and the study described in Example 1.

### Modeling Conclusions

The available population PK model previously developed to describe the efgartigimod concentration in previous studies was refined to be able to adequately capture the PK of the compound+rHuPH20 in the study described in Example 1. More in detail, the absorption model was modified, as the SC treatment groups of efgartigimod+rHuPH20 required the implementation of a sequential zero-first-order process. Furthermore, the administration of efgartigimod with rHuPH20 provided higher relative bioavailability, as compared to the 10 mg/kg SC group in the historical data (0.764 vs. 0.560 for with and without rHuPH20, respectively).

The final PK/total IgG model, previously developed to describe total IgG in the healthy population, consisted of an indirect response model, in which the concentration of efgartigimod stimulated the degradation rate of the biomarker of interest. This model was refined by the inclusion of an effect compartment to adequately capture the total IgG concentration and reduction in healthy volunteers treated with efgartigimod+rHuPH20 in the study described in Example 1. No body weight effect was found to be statistically significant on either PK or PD parameters.

### Simulation Methods and Assumptions

Simulations were performed using R (version 3.4.4, The R foundation for Statistical Computing) and RStudio (version 1.1.463, RStudio Inc, Boston, USA) used in conjunction with a custom-built simulation package.

The PK and PK/total IgG models developed to describe efgartigimod and total IgG concentrations in healthy volunteers from the study described in Example 1 were used to perform simulations. Efgartigimod concentration and total IgG time profiles were simulated based on typical PK and total IgG parameter estimates reported in Table 5 and Table 6, respectively. In addition to the 10 mg/kg IV efgartigimod every week (QW) for 12 weeks scenario, which represented the benchmark for these simulations, different scenarios based on efgartigimod PH20 SC doses ranging between 750 mg and 1750 mg (in 25 mg increments) QW for 12 weeks were simulated. For each scenario, 500 simulations were performed including parameter uncertainty. For the benchmark dose of 10 mg/kg IV QW, a one-hour infusion and a body weight of 70 kg were assumed. For each scenario, the median, 5th, and 95th percentiles of the following three metrics were calculated based on the simulated total IgG concentration-time profiles after efgartigimod administration:
(a) the area under the effect curve for total IgG concentrations after the fourth dose between day 22 and day 29 (AUEC_{D22-D29});
(b) the maximum total IgG reduction after the fourth dose between day 22 and day 29; and
(c) the trough reduction of total IgG on day 29 (i.e., the reduction of total IgG before the dose on day 29 is given).

TABLE 5
Parameter estimates applicable to efgartigimod PH20 SC administration
from efgartigimod PK model for healthy volunteers
Structural Parameters

| | | |
|---|---|---|
| Parameter | Estimate [5% CI; 95% CI] | RSE (%) |

| | | | |
|---|---|---|---|
| CL (L/h) | 0.128 | [0.122; 0.134] | 2.5 |
| V1 (L) | 3.41 | [2.73; 4.09] | 10.2 |
| Q2 (L/h) | 0.00612 | [0.00529; 0.00695] | 6.9 |
| V2 = V3 (L) | 6.74 | [6.16; 7.32] | 4.4 |
| Q3 (L/h) | 0.294 | [0.260; 0.328] | 5.9 |
| Kₐ (1/h) | 0.155 | [0.101; 0.209] | 3.7 |
| F (−) SC | 0.764 | [0.709; 0.818] | 5.1 |
| DUR (h) | 76 | [68.3; 83.7] | 17.7 |

| | | |
|---|---|---|
| Parameter | Value [% CV] | RSE (%) |

Inter-Individual Variability

| | | | |
|---|---|---|---|
| ω²CL | 0.0126 | [11.3%] | 50.6 |
| ω²V1 | 0.554 | [86.0%] | 26.1 |
| ω²V2 = V3 | 0.0758 | [28.1%] | 38.4 |
| ω²F | 0.776 | [108%] | 27.1 |
| ω²DUR | 0.0716 | [27.2%] | 29.4 |

Residual Variability

| | | | |
|---|---|---|---|
| σ² add | 0.0555 | [23.9%] | 12.6 |

TABLE 6
Parameter estimates applicable to efgartigimod PH20 SC administration
from efgartigimod PK/PD model in healthy volunteers

| Parameter | Estimate [5% CI; 95% CI] | | RSE (%) |
|---|---|---|---|
| Baseline IgGt (mg/L) | 8574 | [7786; 9361] | 4.7 |
| Kₒᵤₜ (1/h) | 0.00175 | [0.00132; 0.00218] | 12.6 |
| Eₘₐₓ | 4.70 | FIX^{a} | — |
| EC₅₀ (ng/mL) | 33636 | [22051; 45220] | 17.6 |
| Hill coefficient | 1 | FIX | 28.2 |
| Kₑₒ (1/h) | 0.0288 | [0.0129; 0.0447] | — |

| | | |
|---|---|---|
| Parameter | Value [% CV] | RSE (%) |

Inter-Individual Variability

| | | | |
|---|---|---|---|
| ω²baseline | 0.0704 | [27.0%] | 20.5 |
| ω²EC50 | 0.111 | [34.3%] | 44.6 |

Residual Variability

| | | | |
|---|---|---|---|
| σ² prop | 0.0109 | [10.5%] | 25.2 |

RSE (%) is calculated as standard error/Value*100; % CV is calculated as sqrt(exp(ω²) − 1)*100 or sqrt(exp(σ²) − 1)*100.

### Simulation Results

The median and 5th and 95th percentiles of the metrics obtained with 10 mg/kg IV of efgartigimod QW were: (a) AUEC_{D22-D29}: 949 g h/L (863 g h/L; 1030 g h/L); (b) maximum total IgG reduction after the fourth dose between day 22 and day 29: −66.59% (−68.96%; −64.38%); and (c) trough reduction of total IgG on day 29: −65.75% (−68.43%; −63.42%).

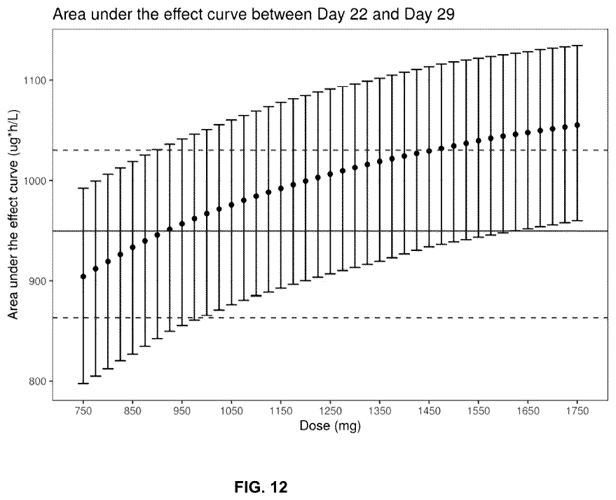

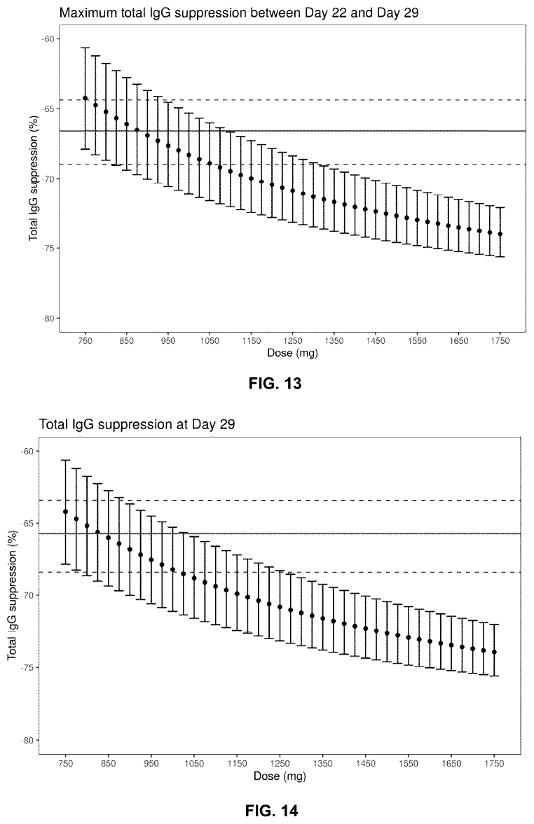

The simulated metrics after administration of different dose levels of efgartigimod PH20 SC are shown in FIGS. 12, 13, and 14, for AUEC_{D22-D29}, maximum total IgG reduction between day 22 and day 29, and total IgG reduction on day 29, respectively. The efgartigimod PH20 SC doses that provided comparable median values to the benchmark scenario for these three metrics were 925 mg (FIG. 12), 900 mg (FIG. 13), and 825 mg (FIG. 14), respectively. These simulations showed SC doses of efgartigimod that are non-inferior to the benchmark IV dose.

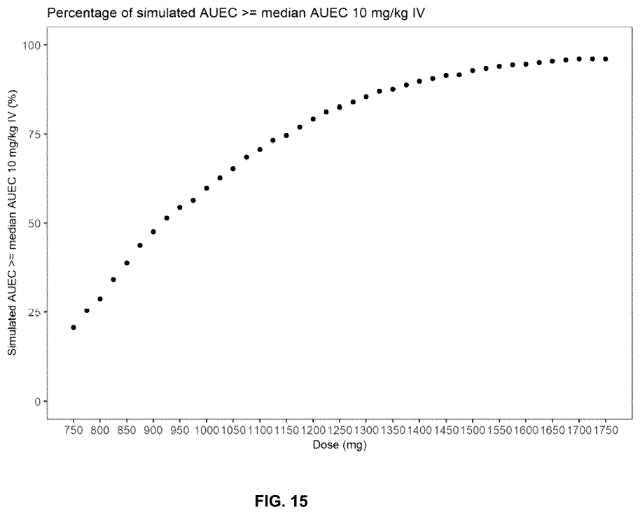

For each dose, the percentage of simulated values exceeding the target level (derived for the benchmark scenario) was calculated for each of the three metrics (see FIGS. 15, 16, and 17). The 825 mg (trough total IgG reduction on day 29), 900 mg (maximum total IgG reduction between day 22 and day 29), and 925 mg (AUEC_{D22-D29}) doses of efgartigimod PH20 SC, provided comparable median values to the benchmark scenario for the three selected metrics.

The 825 mg efgartigimod PH20 SC dose provided 34.2% AUEC_{D22-D29} values above the median AUEC_{D22-D29} obtained with the benchmark scenario, 32.8% of maximum total IgG reduction between day 22 and day 29 below the corresponding median obtained with 10 mg/kg IV of efgartigimod QW, and 46.4% of trough total IgG reduction on day 29 below the corresponding median obtained with the benchmark scenario.

Further, the 900 mg efgartigimod PH20 SC dose provided 47.6% AUEC_{D22-D29} values above the median AUEC_{D22-D29} obtained with the benchmark scenario, 56.4% of maximum total IgG reduction between day 22 and day 29 below the corresponding median obtained with 10 mg/kg IV of efgartigimod QW, and 72.4% of trough total IgG reduction on day 29 below the corresponding median obtained with the benchmark scenario.

Furthermore, the 925 mg efgartigimod PH20 SC dose provided 51.4% AUEC_{D22-D29} values above the median AUEC_{D22-D29} obtained with the benchmark scenario, 65.4% of maximum total IgG reduction between day 22 and day 29 below the corresponding median obtained with 10 mg/kg IV of efgartigimod QW, and 78.4% of trough total IgG reduction on day 29 below the corresponding median obtained with the benchmark scenario.

An overview of the results obtained with the several doses of efgartigimod PH20 SC is shown in Table 7 below.

TABLE 7
Percentage of simulated metrics exceeding the
corresponding median target level obtained
with once weekly 10 mg/kg IV of efgartigimod

| Efgartigimod | | Maximum total | Trough |
|---|---|---|---|
| PH20 SC QW dose | AUEC_{D 22-D 29} | IgG_{D 22-D 29} | total IgG_{D 29} |
| 825 mg | 34.2% | 32.8% | 46.4% |
| 900 mg | 47.6% | 56.4% | 72.4% |
| 925 mg | 51.4% | 65.4% | 78.4% |
| 975 mg | 56.4% | 78.0% | 89.2% |
| 1000 mg | 59.8% | 84.0% | 92.6% |

These results suggested that an efgartigimod PH20 SC dose of at least 975 mg is required to obtain more than 75% values of maximum total IgG reduction between day 22 and day 29 being above the median of maximum total IgG reduction between day 22 and day 29 of the benchmark scenario (see Table 7).

### SC Dose Selection

A dose of 1000 mg of efgartigimod PH20 SC was selected for further clinical development because this dose was predicted to be close to the 5th percentile of the benchmark scenario for AUEC_{D22-D29} and the 95th percentile of the benchmark scenario for the maximum total IgG reduction between day 22 and day 29 and trough total IgG reduction on day 29.

Specifically, the simulations showed that (a) a dose of 1000 mg efgartigimod PH20 SC provided a 5th percentile of AUEC_{D22-D29} comparable to the 5th percentile obtained with 10 mg/kg IV of efgartigimod once per week (FIG. 12); (b) a dose of 950 mg efgartigimod PH20 SC provided a 95th percentile of the maximum total IgG reduction between day 22 and day 29 comparable to the 95th percentile obtained with 10 mg/kg IV of efgartigimod once per week (FIG. 13); and (c) a dose of 900 mg efgartigimod PH20 SC provided a 95th percentile of the trough total IgG reduction on day 29 comparable to the 95th percentile obtained with 10 mg/kg IV of efgartigimod once per week (FIG. 14).

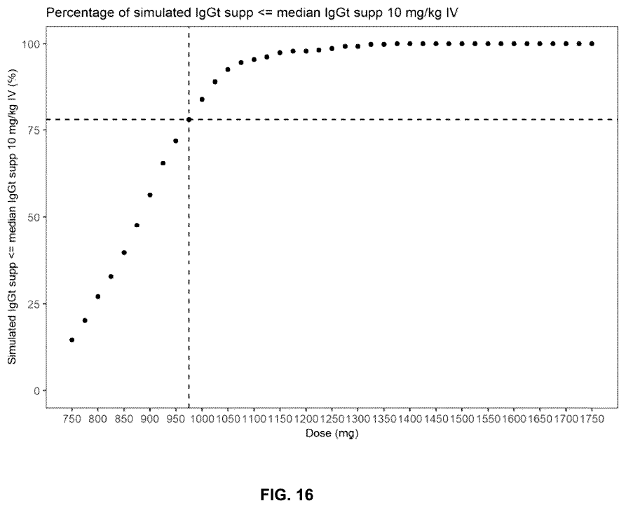

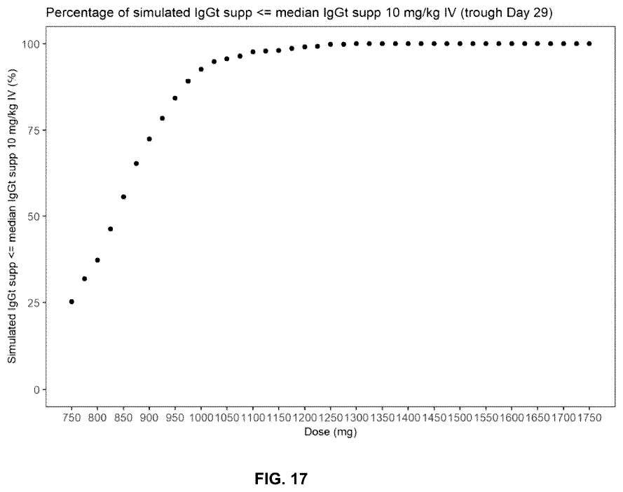

Furthermore, the simulations demonstrated that 1000 mg efgartigimod PH20 SC provided 59.8% AUEC_{D22-D29} values above the median AUEC_{D22-D29} obtained with the benchmark scenario (FIG. 15), 84.0% of maximum total IgG reduction between day 22 and day 29 below the corresponding median obtained with 10 mg/kg IV of efgartigimod once per week (FIG. 16), and 92.6% of trough total IgG reduction on day 29 below the corresponding median obtained with the benchmark scenario of 10 mg/kg IV efgartigimod once per week (FIG. 17) (also see Table 7).

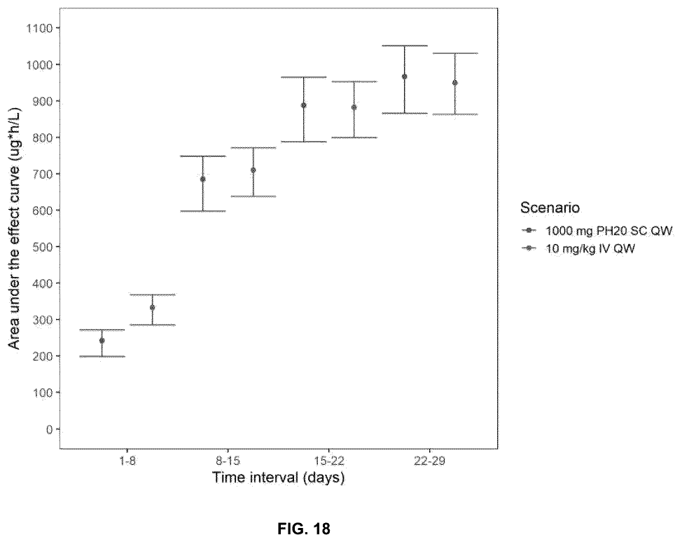

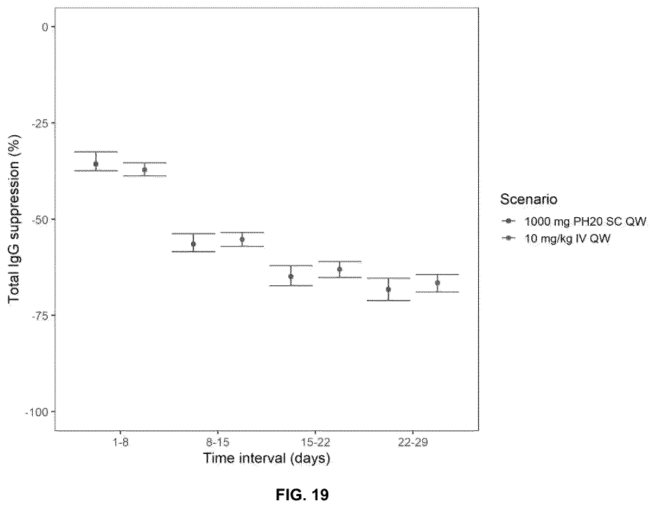

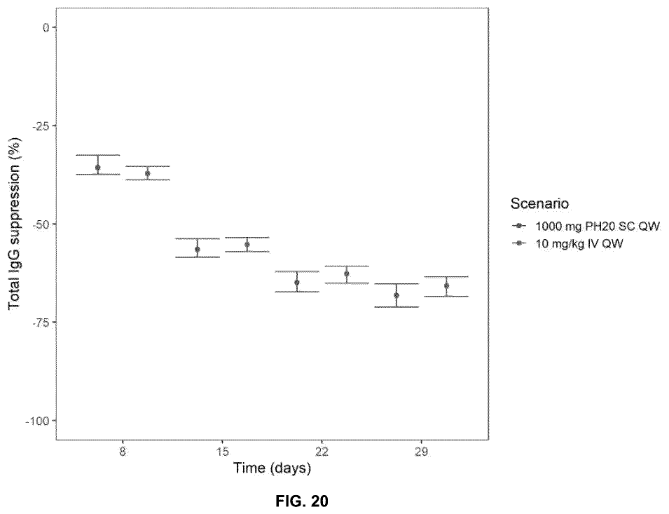

In addition, AUEC (FIG. 18) and maximum total IgG reduction (FIG. 19) obtained with 1000 mg efgartigimod PH20 SC QW and 10 mg/kg IV of efgartigimod QW were calculated between: i) day 1 and day 8; ii) day 8 and day 15; iii) day 15 and day 22; and iv) day 22 and day 29. Total IgG reduction before doses on days 8, 15, 22, and 29, with 1000 mg efgartigimod PH20 SC QW and 10 mg/kg IV of efgartigimod QW were also derived (FIG. 20). The percentages of simulated AUEC obtained with 1000 mg efgartigimod PH20 SC QW above the median AUEC obtained with 10 mg/kg IV of efgartigimod QW in each time interval were predicted to be (FIG. 18): i) 0% (between day 1 and day 8); ii) 25% (between day 8 and day 15); iii) 53.6% (between day 15 and day 22); iv) 59.8% (between day 22 and day 29) (see Table 8).

The percentages of simulated maximum total IgG reduction obtained with 1000 mg efgartigimod PH20 SC QW below the median of the maximum total IgG reduction obtained with 10 mg/kg IV of efgartigimod QW in each time interval were predicted to be (FIG. 19): i) 9.6% (between day 1 and day 8); ii) 78.2% (between day 8 and day 15); iii) 88.4% (between day 15 and day 22); and iv) 84.0% (between day 22 and day 29) (see Table 8). The percentages of simulated total IgG reduction obtained with 1000 mg efgartigimod PH20 SC QW below the median of the total IgG reduction obtained with 10 mg/kg IV of efgartigimod QW were predicted to be: i) 9.6% (before the dose on day 8 is given); ii) 78.2% (before the dose on day 15 is given); iii) 92.0% (before the dose on day 22 is given); iv) 92.6% (before the dose on day 29 is given) (see FIG. 20 and Table 8).

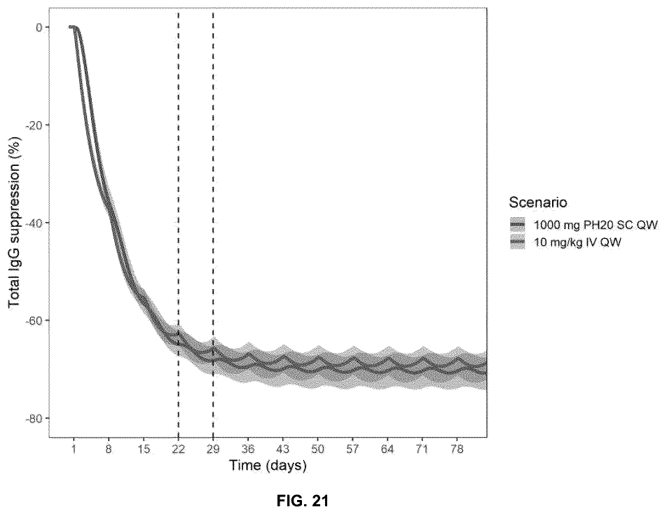

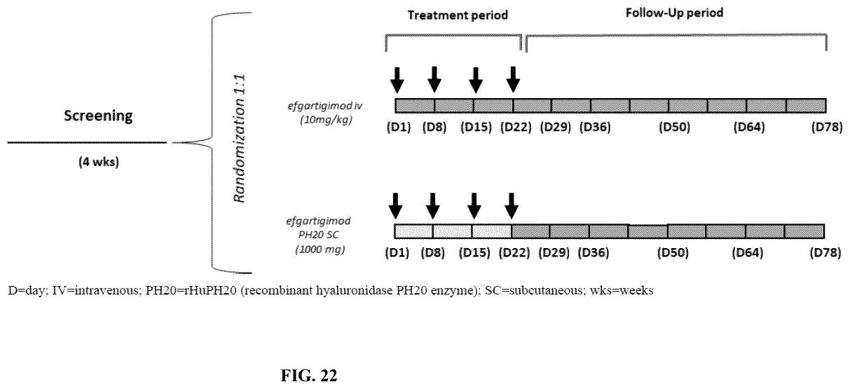

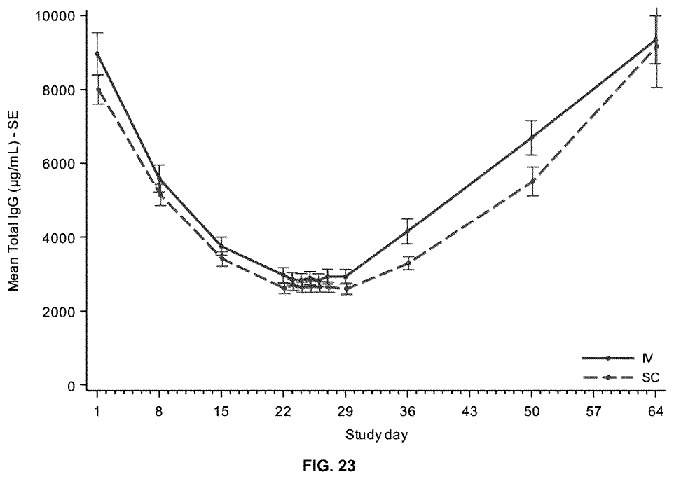

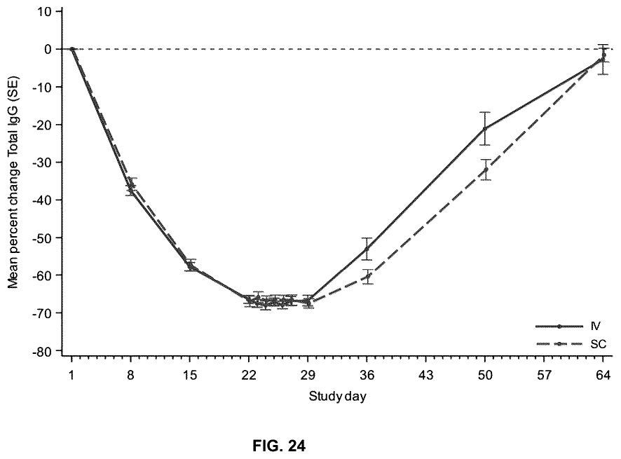

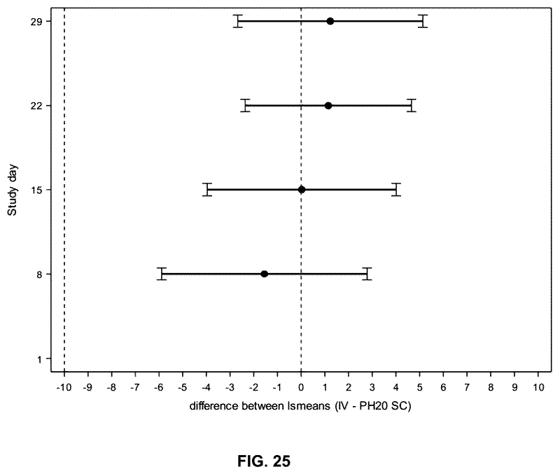

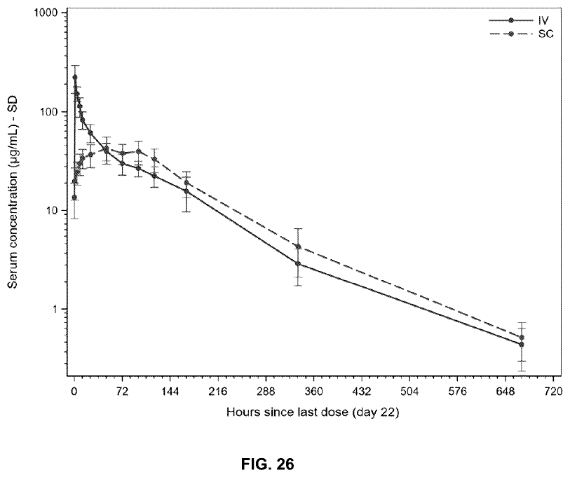

The simulated total IgG profiles obtained with 10 mg/kg IV efgartigimod QW and 1000 mg efgartigimod PH20 SC QW are shown in FIG. 21.

TABLE 8
Percentage of simulated metrics with 1000 mg
efgartigimod PH20 SC QW with respect to the

| Time | % AUEC^{a} ≥ | % maximum total | % trough total |
|---|---|---|---|
| interval | benchmark | IgG ≤ benchmark | IgG ≤ benchmark |
| day 1-8 | 0% | 9.6% | 9.6% |
| day 8-15 | 25.0% | 78.2% | 78.2% |
| day 15-22 | 53.6% | 88.4% | 92.0% |
| day 22-29 | 59.8% | 84.0% | 92.6% |

### Conclusion

Based on the comparable PD parameter of total IgG reduction, a dose of 1000 mg efgartigimod administered subcutaneously with rHuPH20 was proposed for weekly dosing in a clinical trial.

The PK and PK/PD models previously developed to describe efgartigimod and total IgG concentrations in healthy volunteers from the study described in Example 1, were used to perform simulations to support the dose selection of efgartigimod PH20 SC once per week resulting in a similar effect on total IgG as 10 mg/kg IV of efgartigimod once per week. The simulation results suggested that 925 mg, 900 mg, and 825 mg efgartigimod PH20 SC doses provided comparable median AUEC_{D22-D29}, maximum total IgG reduction between day 22 and day 29, and trough total IgG reduction on day 29 to the 10 mg/kg IV efgartigimod QW, respectively.

The 1000 mg dose of efgartigimod PH20 SC was selected for future clinical development because it was predicted to be close to the 5th percentile of the benchmark scenario for AUEC_{D22-D29} and 95^{th} percentile of the benchmark scenario for the maximum total IgG reduction between day 22 and day 29 and trough total IgG reduction on day 29.

### Example 3: A Study to Compare the Pharmacodynamics, Pharmacokinetics, Safety, and Tolerability of Multiple Intravenous Infusions of Efgartigimod with Multiple Subcutaneous Injections of Efgartigimod-PH20 SC in Healthy Subjects

This example describes the protocol and results for a Phase 1 clinical trial to demonstrate that the pharmacodynamic (PD) effect of 4 once-weekly subcutaneous (SC) injections of 1000 mg efgartigimod, co-formulated with rHuPH20 (efgartigimod-PH20), is non-inferior to that of 4 once-weekly intravenous infusions (IV) of efgartigimod at a dose of 10 mg/kg (see the schematic of the study protocol in FIG. 15).

In this study, subjects were randomized in a 1:1 ratio to receive open-label efgartigimod IV or efgartigimod-PH20 SC, respectively. It was assumed that a comparable PD effect would result in comparable efficacy in patients and the non-inferiority of the efgartigimod PD effect of the SC administration compared to the IV administration was investigated.

The efgartigimod IV 10 mg/kg dose selected for this study is the dose that has been shown to be well-tolerated, safe, and associated with clinical efficacy in patients with generalized myasthenia gravis. The EFGARTIGIMOD-PH20 SC 1000 mg dose is predicted to result in a similar PD effect as the efgartigimod IV 10 mg/kg dose, and was chosen based on the modeling and simulations described in Example 2.

### Inclusion and Exclusion Criteria

A total of 54 healthy subjects were randomized in a 1:1 ratio to either efgartigimod IV (27 subjects) or EFGARTIGIMOD-PH20 SC (27 subjects). The subjects were selected based on the inclusion and exclusion criteria listed below.

### Inclusion and Exclusion Criteria

### Inclusion Criteria:

1. The subject is between 18 and 65 years of age, inclusive, on the day when the ICF is signed.
2. The subject is either male or female of non-childbearing potential (postmenopausal [defined by continuous amenorrhea for at least 1 year without an alternative medical cause with a follicle-stimulating hormone (FSH) of >33.4 IU/L; in subjects on hormonal replacement therapy, a historical value pretreatment of >33.4 IU/L will be accepted as proof of menopausal status]) OR have a documented permanent sterilization procedure (i.e., hysterectomy, bilateral salpingectomy, and bilateral oophorectomy).
3. The female subject has a negative pregnancy test at day-1.
4. The subject has a body mass index (BMI) between 18 and 30 kg/m², inclusively, with a weight of ≥50 kg and ≤100 kg at screening.
5. The subject is able to understand the requirements of the study, provide written informed consent (including consent for the use and disclosure of research-related health information), willing and able to comply with the protocol procedures (including required study visits).
6. The subject is in good physical and mental health, per the opinion of the investigator, based on medical history, physical examination, ECG, and vital sign findings; and biochemistry, hematology, virology, and urinalysis test results prior to the first IMP administration.
7. The non-sterilized male subject who is sexually active with a female partner of childbearing potential must use effective contraception. Male subject practicing true sexual abstinence (when consistent with the preferred and usual lifestyle of the participant) can be included. The sterilized male subject who has had a vasectomy with documented aspermia post procedure can be included. In addition, no male subject will be allowed to donate sperm during the period from signing the ICF, throughout the duration of the trial, and 90 days after the last administration of the IMP.
8. The condition of the skin tissue on the subject's abdomen must allow for absorption and assessment of local safety of the planned SC injection, as determined by the investigator.
9. The subject agrees to discontinue and refrain from all medications (including over the counter and/or prescription medications), except for occasional paracetamol use (maximum dose of 2 g/day and maximum of 10 g/2 weeks), antacid use, and ibuprofen use (maximum dose of 400 mg/day and not to be co-administered with antacid), at least 2 weeks before the first efgartigimod administration through the final follow-up visit on day 78.
10. The subject agrees to withhold from strenuous activities from at least 2 weeks before the first efgartigimod administration through the final follow-up visit on day 78.
11. The subject is a non-smoker and does not use any nicotine-containing products. A non-smoker is defined as an individual who has abstained from smoking for at least 1 year prior to screening.
12. The subject has a negative nicotine analyte test at screening and on day-1.
13. The subject has a negative urine drug screen (amphetamines, barbiturates, benzodiazepines, cannabis, cocaine, opiates, methadone, and tricyclic antidepressants) at screening and on day-1.
14. The subject has a negative alcohol urine test at screening and on day-1.
15. The subject has a body temperature of 35.2° C. to 37.6° C. at screening and on day-1.

### Exclusion Criteria:

16. The subject has previously participated in clinical studies with efgartigimod and was administered efgartigimod.
17. The subject has a known hypersensitivity to 1 of the components in the efgartigimod formulation, or a history of severe allergic or anaphylactic reactions, in the opinion of the investigator.
18. The subject tests positively at screening for any of the following conditions
   a. The subject has an active hepatitis B infection (acute or chronic) at screening as determined by hepatitis B serology (https://www.cdc.gov/hepatitis/hbv/pdfs/SerologicChartv8.pdf).
   b. The subject has serology positive for hepatitis C virus antibody (HCV Ab).
   c. The subject has human immunodeficiency virus (HIV) positive serology.
19. Subjects with clinically significant active or chronic uncontrolled bacterial, viral, or fungal infection at screening.
20. Subjects with clinical evidence of other significant serious diseases, subjects who underwent a recent major surgery, or any other reason which could confound the results of the trial or put the subject at undue risk.
21. The subject has total IgG<6 g/L at screening.
22. The subject has presence or sequelae of gastrointestinal, liver, kidney, or any other condition known to potentially interfere with the absorption, distribution, metabolism, or excretion of efgartigimod.
23. The subject has a history of malignancy unless deemed cured by adequate treatment with no evidence of recurrence for ≥3 years before first efgartigimod administration. Subjects with the following cancer can be included anytime:
a. Adequately treated basal cell or squamous cell skin cancer
b. Carcinoma in situ of the cervix
c. Carcinoma in situ of the breast or
d. Incidental histological finding of prostate cancer (TNM stage T1a or T1b)
24. The subject has a clinically relevant abnormality detected on ECG recording regarding either rhythm or conduction (e.g., QTcF>450 ms for male and QTcF>470 ms for female subjects, or a known long QT syndrome). A first-degree heart block or sinus arrhythmia will not be considered a significant abnormality.
25. The subject has clinically relevant abnormalities detected in vital sign measurements prior to dosing.
26. The subject has significant blood loss (including blood donation >500 mL) or has had a transfusion of any blood product within 12 weeks prior to the (first) efgartigimod administration or a scheduled transfusion within 4 weeks after the end of the study.
27. The subject has been treated with any drug known to have a well-defined potential for toxicity to a major organ in the last 3 months preceding the initial efgartigimod administration.
28. The subject has a history of consuming more than 21 units of alcoholic beverages per week or a history of alcoholism or drug/chemical/substance abuse within 2 years prior to screening (Note: 1 unit=330 mL of beer, 110 mL of wine or 28 mL of spirits). Regular consumption of a large quantity of coffee, tea (>6 cups per day), or equivalent within 3 weeks prior to first dose is also exclusionary.
29. The subject has received investigational drug within 3 months or 5 half-lives of the drug (whichever is longer) prior to first efgartigimod administration.
30. The subject has received a vaccination (e.g., influenza vaccine) within the last 4 weeks prior to screening.
31. The subject has received any systemic immunosuppressant agent within 6 months prior to the initial efgartigimod administration.
32. The subject has received any systemic steroid within 3 months prior to the initial efgartigimod administration.
33. The subject has received any monoclonal antibody, within 6 months prior to first efgartigimod administration.
34. The subject is an employee of the investigator or study center, with direct involvement in the proposed study or other studies under the direction of that investigator or study center, as well as a family member of an employee or the investigator.
35. The subject has any condition or circumstances that in the opinion of the investigator may make a subject unlikely or unable to complete the study or comply with study procedures and requirements.
36. The subject has any condition impairing phlebotomy.
37. The subject is a pregnant or lactating women or intending to become pregnant during the study or within 90 days after last dosing.
38. The subject has a positive nasopharyngeal PCR test for SARS-CoV-2 on days-2 or -1.
39. The subject has had any contact with SARS-CoV-2 positive or COVID-19 patients within the last 2 weeks prior to admission to the clinical research center.

### Investigational Product, Dosage, and Mode of Administration

The efgartigimod IV product is a 20R vial with an extractable volume of 20 mL. One vial can deliver 400 mg efgartigimod.

The efgartigimod-PH20 SC product is a 10R vial with an extractable volume of 10 mL, at a concentration of 165 mg/mL. The vial is ready to use and can deliver 1650 mg efgartigimod.

### Concomitant Therapy

From 2 weeks prior to the first administration of efgartigimod until the end of the study, subjects are not allowed to use any kind of procedure or medication (including over-the-counter and/or prescription medication, dietary supplements, nutraceuticals, vitamins and/or herbal supplements such as Ginkgo biloba or St. John's wort), except occasional paracetamol use (maximum dose of 2 g/day and maximum of 10 g/2 weeks), antacid use, and ibuprofen use (maximum dose of 400 mg/day and not to be co-administered with antacid), after consultation of, and approval by the investigator.

All medications taken from receipt of the informed consent signature until the end of the study or started during the course of the study will be recorded.

Any medications started, stopped, up-titrated, or down-titrated in response to an AE will also be recorded.

### Objectives and Endpoints

The primary objective of the study is to demonstrate that the PD effect of 4 once-weekly SC injections of 1000 mg efgartigimod-PH20 is non-inferior to that of 4 once-weekly intravenous infusions (IV) of efgartigimod at a dose of 10 mg/kg by comparing the percentage reduction in total immunoglobulin G (IgG) levels after 4 weeks (day 29), i.e., 1 week after the fourth administration, using a non-inferiority margin of 10%.

The secondary objectives of the study are:
to compare the PD effect of efgartigimod IV and efgartigimod-PH20 SC over time;
to evaluate the PK of efgartigimod IV and efgartigimod-PH20 SC; and
to evaluate the safety, tolerability, and anti-drug antibodies (ADA) of efgartigimod IV and efgartigimod-PH20 SC.

The primary endpoint of the study is the percentage reduction in total IgG levels, compared to baseline, at day 29 (week 4), 7 days after the fourth IV or SC administration of efgartigimod.

The secondary endpoints of the study are:
percentage reduction in total IgG levels at all other assessment timepoints as of week 4;
percentage reduction in levels of IgG subtypes (IgG1, IgG2, IgG3, and IgG4) at all assessment timepoints;
absolute values and changes from baseline in total IgG levels and levels of IgG subtypes (IgG1, IgG2, IgG3, and IgG4) at all assessment timepoints;
AUEC for percentage reduction in total IgG levels and for each subtype per weekly interval after each dose (week 1, week 2, week 3, and week 4), over the interval week 1 to week 4, and over the entire study period (week 1 to week 11);
serum levels of efgartigimod and derived PK parameters; and
clinical laboratory evaluations, vital sign measurements, ECG recordings, and incidence and characterization of TEAEs.

### Sample Collection and Analysis

### Pharmacokinetics/Pharmacodynamics

Efgartigimod concentration in serum was determined using a validated enzyme-linked immunosorbent assay (ELISA). The lower limit of quantification (LLOQ) was 300 ng/mL. Concentrations were calculated by interpolation from a calibration curve. Quality control samples were analyzed throughout the study. Their measured concentrations were used to determine between-run, overall precision, and accuracy of the analyses.

Blood samples were on study days 1, 8, 15, 22, 23-27, 29, 36, 50, 64, and 78 (taken prior to each IV or SC efgartigimod administration on treatment days) to determine levels of total IgG and IgG subtypes (IgG1, IgG2, IgG3, and IgG4).

### Anti-Drug Antibody (ADA) Assessment

For subjects in the SC treatment group, individual serum and plasma titers of ADAs against efgartigimod and rHuPH20, respectively, were measured before and after the SC injection of efgartigimod-PH20. For subjects in the IV treatment group, individual serum titers of ADAs against efgartigimod were measured before and after IV infusion of efgartigimod.

Samples for ADA determination were taken on study days 1, 15, 29, 50, and 78.

### Primary Endpoint Analysis Based on PD Analysis

The primary endpoint was defined as the percentage reduction in total IgG levels, compared to baseline, at day 29 (week 4), i.e., 7 days after the fourth IV or SC administration of efgartigimod.

The hypotheses for the evaluation of the non-inferiority, with a non-inferiority margin of 10%, comparing SC administration with the IV administration were:

$$H 0 : {μ}_{iv} - {μ}_{sc} \geq 10$$

$$H 1 : {μ}_{iv} - {μ}_{sc} < 10$$

μᵢᵥ and μ_{sc} are the estimated averages in % reduction of total IgG after 4 weeks (day 29) in the group of subjects receiving efgartigimod as IV or SC administration, respectively.

An analysis of covariance model (ANCOVA) was used to estimate the average percentage reduction at week 4 for each treatment group as well as the 2-sided 95% CI for the difference between both treatment groups. The model included a factor for treatment and the baseline IgG value as covariate.

When the upper limit of the 95% CI (mean reduction with IV—mean reduction with SC) was below the margin of 10% the SC formulation was considered non-inferior to the IV formulation.

### Primary Endpoint Analysis Based on PD Analysis

The secondary PD endpoints included:
Percentage reduction in total IgG levels at all other assessment timepoints as of week 4
Percentage reduction in levels of IgG subtypes (IgG1, IgG2, IgG3, and IgG4) at all assessment timepoints
Absolute values and changes from baseline in total IgG levels and levels of IgG subtypes (IgG1, IgG2, IgG3, and IgG4) at all assessment timepoints
AUEC for percentage reduction in total IgG levels and for each subtype per weekly interval after each dose (week 1, week 2, week 3, and week 4), over the interval week 1 to week 4, and over the total study period (week 1-week 11).

The same ANCOVA model was used for all secondary endpoints. All endpoints were summarized per time point or interval and per treatment group.

### Results

### Pharmacodynamics (PD)

An interim analysis of data from the study shows that the absolute values of total IgG and percent changes from baseline in IgG levels over time for the efgartigimod-PH20 SC and efgartigimod IV groups are presented in FIG. 16 and FIG. 17, respectively.

The pattern of total IgG reduction is comparable between both treatment groups, achieving a maximum reduction approximately 1 week after last administration. Thereafter, mean total IgG slowly increased and returned to baseline by day 64 (i.e., 42 days after last administration). It should be noted that due to the data cutoff, the number of observations after day 29 gradually decreases (see Table 9).

TABLE 9
Summary statistics for the percent change from baseline
in total IgG following 4 weekly 1000 mg efgartigimod-
PH20 SC and 10 mg/kg efgartigimod IV doses

| | | | | | | | Mini- | Maxi- |
|---|---|---|---|---|---|---|---|---|
| Arm | Study | N | Mean | Median | SD | SE | mum | mum |
| IV | 8 | 23 | −37.52 | −37.37 | 6.23 | 1.3 | −49.3 | −24.54 |
| | 15 | 23 | −57.74 | −57.14 | 5.43 | 1.13 | −72.28 | −46.57 |
| | 22 | 23 | −66.46 | −67.02 | 5.07 | 1.06 | −74.65 | −55.14 |
| | 23 | 23 | −67.53 | −68.17 | 4.88 | 1.02 | −75.7 | −54.81 |
| | 24 | 23 | −68.04 | −68.79 | 5.62 | 1.2 | −77.79 | −58.15 |
| | 25 | 23 | −67.18 | −67.31 | 4.9 | 1.02 | −77.21 | −55.6 |
| | 26 | 23 | −67.95 | −68.07 | 4.79 | 1 | −76.57 | −55.6 |
| | 27 | 23 | −66.89 | −67.54 | 5.87 | 1.22 | −76.21 | −53.39 |
| | 29 | 23 | −66.77 | −68.57 | 6.81 | 1.42 | −75.29 | −45.04 |
| | 36 | 23 | −53.01 | −57.02 | 13.91 | 2.9 | −69.71 | −7 |
| | 50 | 13 | −21.11 | −23.67 | 15.83 | 4.39 | −37.48 | 4.32 |
| | 64 | 6 | −2.74 | −2.92 | 9.7 | 3.96 | −13.33 | 12.52 |
| | 8 | 21 | −35.87 | −36.7 | 7.55 | 1.65 | −45.58 | −14.84 |
| | 15 | 21 | −57.35 | −58.54 | 7.34 | 1.6 | −68.38 | −40.51 |
| | 22 | 21 | −66.99 | −69.57 | 6.45 | 1.41 | −76.62 | −52.81 |
| | 23 | 21 | −65.89 | −67.05 | 6.69 | 1.46 | −73.85 | −47.86 |
| PH20 | 24 | 21 | −66.76 | −67.42 | 5.64 | 1.23 | −76.18 | −57.44 |
| SC | 25 | 21 | −66.65 | −67.52 | 6.26 | 1.37 | −75.22 | −54.6 |
| | 26 | 21 | −66.59 | −67.02 | 5.6 | 1.22 | −76.52 | −57.7 |
| | 27 | 21 | −66.63 | −67.74 | 6.29 | 1.37 | −76.87 | −56.38 |
| | 29 | 21 | −67.51 | −69.44 | 5.72 | 1.25 | −75.21 | −55.61 |
| | 36 | 17 | −60.41 | −62.17 | 7.91 | 1.92 | −70.54 | −48.25 |
| | 50 | 14 | −32.02 | −32.21 | 10.08 | 2.69 | −46.62 | −13.65 |
| | 64 | 4 | −1.56 | −2.79 | 3.59 | 1.8 | −4.39 | 3.7 |

The primary endpoint for this study was defined as the percentage reduction in total IgG from baseline, 1 week after the fourth administration of study medication (i.e., day 29). To derive a confidence interval (CI) for the difference in the percent change from baseline in total IgG between the 2 treatment arms, analysis of covariance (ANCOVA) was used, including a factor for treatment arm and the baseline IgG as covariate. From this model, 95% 2-sided CIs were derived for the difference in percent change from baseline for day 29 and the preceding weekly visits.

Based on this model, the difference in reduction of total IgG at day 29 was 1.23 percentage points (PP) (see Table 10), meaning a slightly higher decrease in total IgG with efgartigimod-PH20 SC dosing as compared to efgartigimod IV dosing. Although the non-inferiority evaluation was not the objective of the interim analyses, the results are satisfying the non-inferiority criteria: the lower limit (−2.68 PP) of the 95% CI of the difference between the treatment arms at day 29 was already above the prespecified non-inferiority margin of −10%. In fact, the lower limits of the confidence intervals for the differences in reduction of total IgG at days 8, 15, and 22 were all found to be above this prespecified non-inferiority margin (see FIG. 18 and Table 10).

TABLE 10
Summary statistics for the comparison of percent change
from baseline in total IgG between the efgartigimod
IV and efgartigimod-PH20 SC treatment arms (IV-PH20
SC) for the first 4 weeks of treatment

| | | Difference | |
|---|---|---|---|
| | Lower | % Change Total IgG | Upper |
| Study day | CL | (IV-PH20 SC) | CL |
| 8 | −5.8922 | −1.554 | 2.7837 |
| 15 | −3.9677 | 0.023 | 4.0146 |
| 22 | −2.3643 | 1.146 | 4.6566 |
| 29 (primary endpoint) | −2.6777 | 1.230 | 5.1372 |

The results from the interim analysis suggest that the effect of 4 weekly SC injections of 1000 mg efgartigimod-PH20 SC, on the percent change from baseline in total IgG up to day 29, is not inferior to the effect of 4 weekly IV infusions with 10 mg/kg efgartigimod IV.

After efgartigimod-PH20 SC and efgartigimod IV administration the mean percent change from baseline in total IgG levels decreased after each dose of efgartigimod, up to a maximum reduction of 67.5% at day 29 (7 days after last injection) and of 68.0% at day 26 (4 days after last infusion), respectively.

Baseline levels of total IgG, as well as levels at time of maximum reduction were comparable between both treatment groups, i.e., 8003 μg/mL and 8968 μg/mL at baseline, and 2600 μg/mL and 2829 μg/mL at time of maximal reduction after efgartigimod-PH20 SC and efgartigimod IV, respectively (see Table 11).

TABLE 11
Summary statistics total IgG (ug/mL) over time

| | | | | | | | |
|---|---|---|---|---|---|---|---|
| Study | | | | | | | |
| day | N | Mean | Median | SD | SE | Minimum | Maximum |

IV

| | | | | | | | |
|---|---|---|---|---|---|---|---|
| 1 | 23 | 8968.26 | 8750 | 2763.82 | 576.3 | 4100 | 14300 |
| 8 | 23 | 5584.78 | 5550 | 1761.56 | 367.31 | 2510 | 9110 |
| 15 | 23 | 3751.3 | 3610 | 1176.87 | 245.39 | 2060 | 6000 |
| 22 | 23 | 2971.3 | 2780 | 962.59 | 200.71 | 1610 | 5450 |
| 23 | 23 | 2860.43 | 2670 | 847.81 | 176.78 | 1600 | 4610 |
| 24 | 22 | 2827.27 | 2555 | 875 | 186.55 | 1650 | 4750 |
| 25 | 23 | 2890.87 | 2730 | 863.38 | 180.03 | 1470 | 4880 |
| 26 | 23 | 2829.13 | 2730 | 843.18 | 175.81 | 1400 | 4500 |
| 27 | 23 | 2929.57 | 2790 | 961.46 | 200.48 | 1520 | 5360 |
| 29 | 23 | 2928.26 | 2710 | 940.71 | 196.15 | 1560 | 4750 |
| 36 | 23 | 4153.48 | 3630 | 1624.64 | 338.76 | 2030 | 7970 |
| 50 | 13 | 6684.62 | 6540 | 1684.35 | 467.16 | 4240 | 9710 |
| 64 | 6 | 9345 | 9985 | 1591.7 | 649.81 | 7280 | 11000 |

SC-PH20

| | | | | | | | |
|---|---|---|---|---|---|---|---|
| 1 | 21 | 8002.86 | 7860 | 1830.85 | 399.52 | 4980 | 11500 |
| 8 | 21 | 5140.95 | 5030 | 1310.03 | 285.87 | 2710 | 8030 |
| 15 | 21 | 3407.62 | 3550 | 899.81 | 196.35 | 1750 | 4730 |
| 22 | 21 | 2610.48 | 2720 | 640.55 | 139.78 | 1500 | 3530 |
| 23 | 21 | 2703.81 | 2780 | 683.99 | 149.26 | 1530 | 3900 |
| 24 | 21 | 2639.52 | 2650 | 647.62 | 141.32 | 1560 | 3660 |
| 25 | 21 | 2661.9 | 2690 | 737.64 | 160.97 | 1400 | 4060 |
| 26 | 21 | 2650.95 | 2710 | 645.14 | 140.78 | 1580 | 3760 |
| 27 | 21 | 2642.86 | 2720 | 640.15 | 139.69 | 1470 | 3480 |
| 29 | 21 | 2600.48 | 2700 | 699.1 | 152.56 | 1450 | 3650 |
| 36 | 17 | 3292.94 | 3500 | 725.06 | 175.85 | 2140 | 4490 |
| 50 | 14 | 5507.14 | 5415 | 1464.33 | 391.36 | 3570 | 9670 |
| 64 | 4 | 9170 | 9400 | 2230.61 | 1115.3 | 6680 | 11200 |

### Pharmacokinetics (PK)

The PK profile after the fourth weekly administration of 1000 mg efgartigimod-PH20 SC or 10 mg/kg efgartigimod IV is presented in FIG. 19 and PK parameters are summarized in Table 12. For this interim evaluation, the PK parameters were estimated based on scheduled sampling times.

TABLE 12
Summary statistics of efgartigimod PK parameters after
a fourth weekly administration of 10 mg/kg efgartigimod
IV or 1000 mg efgartigimod-PH20 SC in healthy subjects

| | efgartigimod IV | | efgartigimod-PH20 SC | |
|---|---|---|---|---|
| | 10 mg/kg | | 1000 mg | |
| n | 23 | | 21 | |
| C_{trough} (μg/mL), mean (SD) | 13.6 | (5.32) | 19.9 | (7.11) |
| Cₘₐₓ (μg/mL), mean (SD) | 225 | (69.7) | 46.6 | (11.9) |
| tₘₐₓ (h), median (min-max) | 1.0 | (1.0-4.0) | 48.0 | (8.0-96.0) |
| AUC_{0-168 h} (μg · h/mL), | 6664 | (1085) | 5699 | (1278) |
| mean (SD) | | | | |
| t_{1/2} (h), mean (SD) | 75.6 | (13.2) | 83.2 | (16.3) |

After multiple injections of 1000 mg efgartigimod-PH20 SC, a plateau phase consisting of 1 or more peaks was observed between 24 and 120 hours post dose, suggesting a prolonged absorption phase due to the SC route of administration. Median tₘₐₓ was 48 hours with individual values ranging between 8 and 96 hours. Mean (SD) efgartigimod C_{trough} and Cₘₐₓ after the fourth SC injection were 19.9 (7.11) μg/mL and 46.6 (11.9) μg/mL, respectively.

Based on mean values, Cₘₐₓ and AUC₀₋₁₆₈ₕ were approximately 80% and 15% lower, respectively, while C_{trough} was approximately 50% higher after 1000 mg efgartigimod-PH20 SC compared with 10 mg/kg efgartigimod IV. The apparent elimination half-life (t_{1/2}) was comparable with mean (SD) values of 83.2 (16.3) hours and 75.6 (13.2) hours after 1000 mg efgartigimod-PH20 SC and 10 mg/kg efgartigimod IV, respectively.

### Conclusion

The 1000 mg efgartigimod-PH20 SC fixed dose results in a similar total IgG reduction, and is therefore, non-inferior to the 10 mg/kg efgartigimod IV dose. This was surprising because, if a classic PK model was used to calculate an SC dose of efgartigimod with comparable bioavailability to the effective IV dose, the dose would have been double the weight-based IV dose (the bioavailability of efgartigimod SC is about 47% that of efgartigimod IV). Instead, the PK/PD modelling approach, based on matching PD parameters to a reference IV dose, described in Example 2 identified a fixed dose that is safe and effective, and will likely lead to increased patient compliance.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A unit dosage form for subcutaneous administration of a biologic, wherein:
(a) said biologic has an RDᵢᵥ, which results in a PKᵢᵥ and a PDᵢᵥ in a subject upon intravenous administration;
(b) said unit dosage form comprises an RD_{sc} of the biologic, which results in a PK_{sc} and a PD_{sc} in a subject upon subcutaneous administration; and
(c) the ratio PK_{sc}/PKᵢᵥ is less than 0.8 and the ratio PD_{sc}/PDᵢᵥ is from 0.9 to 1.1.

2-4. (canceled)

5. A unit dosage form for subcutaneous administration of a biologic, wherein:
(a) the biologic has an RDᵢᵥ, which results in a PKᵢᵥ and a BLᵢᵥ in a subject upon intravenous administration;
(b) the unit dosage form comprises an RD_{sc} of the biologic, which results in a PK_{sc} and a BL_{sc} in a subject upon subcutaneous administration; and
(c) the ratio PK_{sc}/PKᵢᵥ is less than about 0.8 and the ratio BL_{sc}/BLᵢᵥ is of about 0.9 to about 1.1.

6. A unit dosage form for subcutaneous administration of a biologic, wherein the amount subcutaneous dose of the biologic in the unit dosage form was determined by a method comprising the steps of:
(a) administering a subcutaneous dose of the biologic to a subject, wherein the biologic has an RDᵢᵥ, which results in a PKᵢᵥ and a BLᵢᵥ;
(b) determining the BL_{sc} of the biologic;
(c) determining the PK_{sc} of the biologic; and
(d) determining a subcutaneous dose that would result in a BL_{sc}/BLᵢᵥ ratio of about 0.9 to about 1.1 and a PK_{sc}/PKᵢᵥ ratio less than about 0.8.

7-29. (canceled)

30. A method of determining a therapeutically effective dose of a biologic for subcutaneous administration, the method comprising:
(a) administering a subcutaneous dose of the biologic to a subject, wherein the biologic has an RDᵢᵥ, which results in a PKᵢᵥ and a BLᵢᵥ;
(b) determining a BL_{sc} of the biologic;
(c) determining a PK_{sc} of the biologic; and
(d) determining a subcutaneous dose that would result in a BL_{sc}/BLᵢᵥ ratio of about 0.9 to about 1.1 and a PK_{sc}/PKᵢᵥ ratio less than about 0.8,
thereby determining a therapeutically effective dose of the biologic for subcutaneous administration.

31. The method of claim 30, wherein the subject is a healthy volunteer or a non-human animal.

32. A method of treating a subject with a subcutaneous dose of a biologic, wherein the subcutaneous dose of the biologic is determined by a method comprising the steps of:
(a) administering a subcutaneous dose of the biologic to a subject, wherein the biologic has an RDᵢᵥ, which results in a PKᵢᵥ and a BLᵢᵥ;
(b) determining a BL_{sc} of the biologic;
(c) determining a PK_{sc} of the biologic; and
(d) determining a subcutaneous dose that would result in a BL_{sc}/BLᵢᵥ ratio of about 0.9 to about 1.1 and a PK_{sc}/PKᵢᵥ ratio less than about 0.8.

33-36. (canceled)

37. The method of claim 32, wherein the BL_{sc} and the BLᵢᵥ are levels of total IgG in a serum sample of the subject.

38-42. (canceled)

43. The method of claim 32, wherein the biologic comprises or consists of a variant Fc region, or FcRn binding fragment thereof, which binds to FcRn with a higher affinity at pH5.5 as compared to a corresponding wild-type Fc region.

44. (canceled)

45. The method of claim 43, wherein the biologic is efgartigimod.

46. The method of claim 45, wherein the RDᵢᵥ is 10 mg/kg.

47. The method of claim 45, wherein the RDᵢᵥ is 25 mg/kg.

48. The method of claim 32, wherein the therapeutically effective amount of the biologic is co-administered with a hyaluronidase enzyme.

49. The method of claim 32, wherein the therapeutically effective amount of the biologic is administered before or after a hyaluronidase enzyme.

50. The method of claim 48, wherein the hyaluronidase enzyme comprises an amino acid sequence selected from the group consisting of SEQ ID NO: 5-96.

51. The method of claim 48, wherein the hyaluronidase enzyme is rHuPH20.

52. The method of claim 48, wherein the amount of hyaluronidase enzyme is from 1000 U/ml to 3000 U/ml, preferably 2000 U/mL.

53. A method of treating myasthenia gravis in a human patient, comprising:
administering a variant Fc region, or FcRn binding fragment thereof, subcutaneously to the patient as a weekly dose of between 950 and 1050 mg, independent of the weight of the patient, wherein
a total serum IgG reduction in the patient of at least 60% compared to baseline IgG level is obtained, and wherein the Fc domains of the variant Fc region comprise the amino acids Y, T, E, K, F, and Y at EU Kabat positions 252, 254, 256, 433, 434, and 436, respectively.

54. The method of claim 53, wherein the weekly dose is about 1000 mg.

55. A method of treating pemphigus vulgaris in a human patient, comprising:
administering a variant Fc region, or FcRn binding fragment thereof, subcutaneously to the patient as a weekly dose of between 1950 and 2050 mg, independent of the weight of the patient, wherein
a total serum IgG reduction in the patient of at least 60% compared to baseline IgG level is obtained, and wherein the Fc domains of the variant Fc region comprise the amino acids Y, T, E, K, F, and Y at EU Kabat positions 252, 254, 256, 433, 434, and 436, respectively.

56. The method of claim 55, wherein the weekly dose is about 2000 mg.

57. The method of claim 53, wherein the treatment comprises at least 4 weekly doses.

58. The method of claim 53, wherein the variant Fc region, or FcRn binding fragment thereof is administered with a hyaluronidase enzyme.

59. The method of claim 58, wherein the variant Fc region, or FcRn binding fragment thereof is administered before or after the hyaluronidase enzyme.

60. The method of claim 58, wherein the hyaluronidase enzyme comprises an amino acid sequence selected from the group consisting of SEQ ID NO: 5-96.

61. The method of claim 58, wherein the hyaluronidase enzyme is rHuPH20.

62. The method of claim 53, wherein the percentage of total serum IgG reduction is achieved within 1 month from the first dose.

63. The method of claim 53, wherein the maximum percentage of total serum IgG reduction is achieved within 1 month from the first dose.

64. The method of claim 53, wherein the total IgG level is reduced to 2500 to 3500 μg/mL.

65. The method of claim 53, wherein the total serum IgG in the patient is analyzed using a bioanalytical method, preferably ELISA or automated diagnostic analyzer (IVD).

66. The method of claim 53, wherein at least one of the subtypes of IgG is reduced.

67. The method of claim 53, wherein the variant Fc region is efgartigimod.


SEQUENCE LISTING (96 sequences)

SEQ ID NO: 1 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 2 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - protein, 509 aa, Homo sapiens
  source 1..509: note = Precursor Full-length human PH20, signal sequence 1-35
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 5 - protein, 447 aa, Homo sapiens [sequence omitted]

SEQ ID NO: 6 - protein, 430 aa, Homo sapiens
  source 1..430: note = Mature human PH20 36-465
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 7 - protein, 431 aa, Homo sapiens
  source 1..431: note = mature human PH20 36-466
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCID

SEQ ID NO: 8 - protein, 432 aa, Homo sapiens
  source 1..432: note = mature human PH20 36-467
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 9 - protein, 433 aa, Homo sapiens
  source 1..433: note = mature human PH20 36-468
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 10 - protein, 434 aa, Homo sapiens
  source 1..434: note = mature PH20 36-469
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFL

SEQ ID NO: 11 - protein, 435 aa, Homo sapiens
  source 1..435: note = mature human PH20 36-470
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 12 - protein, 436 aa, Homo sapiens
  source 1..436: note = mature human PH20 36-471
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKP

SEQ ID NO: 13 - protein, 437 aa, Homo sapiens
  source 1..437: note = mature human PH20 36-472
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPP

SEQ ID NO: 14 - protein, 438 aa, Homo sapiens
  source 1..438: note = mature human PH20 36-473
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPM

SEQ ID NO: 15 - protein, 439 aa, Homo sapiens
  source 1..439: note = mature human PH20 36-474
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPME

SEQ ID NO: 16 - protein, 440 aa, Homo sapiens
  source 1..440: note = mature human PH20 36-475
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMET

SEQ ID NO: 17 - protein, 441 aa, Homo sapiens
  source 1..441: note = mature human PH20 36-476
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETE

SEQ ID NO: 18 - protein, 442 aa, Homo sapiens
  source 1..442: note = mature human PH20 36-477
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEE

SEQ ID NO: 19 - protein, 443 aa, Homo sapiens
  source 1..443: note = mature human PH20 36-478
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 20 - protein, 444 aa, Homo sapiens
  source 1..444: note = mature human PH20 36-479
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ

SEQ ID NO: 21 - protein, 445 aa, Homo sapiens
  source 1..445: note = mature human PH20 36-480
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 22 - protein, 446 aa, Homo sapiens
  source 1..446: note = mature human PH20 36-481
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 23 - protein, 448 aa, Homo sapiens
  source 1..448: note = mature human PH20 36-483
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 24 - protein, 449 aa, Homo sapiens
  source 1..449: note = mature human PH20 36-484
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNA

SEQ ID NO: 25 - protein, 450 aa, Homo sapiens
  source 1..450: note = mature human PH20 36-485
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNAS

SEQ ID NO: 26 - protein, 451 aa, Homo sapiens
  source 1..451: note = mature human PH20 36-486
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASP

SEQ ID NO: 27 - protein, 452 aa, Homo sapiens
  source 1..452: note = mature human PH20 36-487
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPS

SEQ ID NO: 28 - protein, 453 aa, Homo sapiens
  source 1..453: note = mature human PH20 36-488
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPST

SEQ ID NO: 29 - protein, 454 aa, Homo sapiens
  source 1..454: note = mature human PH20 36-489
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTL

SEQ ID NO: 30 - protein, 455 aa, Homo sapiens
  source 1..455: note = mature human PH20 36-490
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 31 - protein, 456 aa, Homo sapiens
  source 1..456: note = mature human PH20 36-491
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSA

SEQ ID NO: 32 - protein, 457 aa, Homo sapiens
  source 1..457: note = mature human PH20 36-492
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSAT

SEQ ID NO: 33 - protein, 458 aa, Homo sapiens
  source 1..458: note = mature human PH20 36-493
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATM

SEQ ID NO: 34 - protein, 459 aa, Homo sapiens
  source 1..459: note = mature human PH20 36-494
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMF

SEQ ID NO: 35 - protein, 460 aa, Homo sapiens
  source 1..460: note = mature human PH20 36-495
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFI

SEQ ID NO: 36 - protein, 461 aa, Homo sapiens
  source 1..461: note = mature human PH20 36-496
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIV

SEQ ID NO: 37 - protein, 462 aa, Homo sapiens
  source 1..462: note = mature human PH30 36-497
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVS

SEQ ID NO: 38 - protein, 463 aa, Homo sapiens
  source 1..463: note = mature human PH20 36-498
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSI

SEQ ID NO: 39 - protein, 464 aa, Homo sapiens
  source 1..464: note = mature human PH20 36-499
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSIL

SEQ ID NO: 40 - protein, 465 aa, Homo sapiens
  source 1..465: note = mature human PH20 36-500
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILF

SEQ ID NO: 41 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 42 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTSGALLCSQALCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 43 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPFILNVTSGALLCSQALCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 44 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEYMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 45 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 46 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 47 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSNTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 48 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 49 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 50 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 51 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTSGAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 52 - protein, 453 aa, synthetic construct
  source 1..453: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 53 - protein, 430 aa, synthetic construct
  source 1..430: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 54 - protein, 433 aa, synthetic construct
  source 1..433: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 55 - protein, 436 aa, synthetic construct
  source 1..436: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKP

SEQ ID NO: 56 - protein, 452 aa, synthetic construct
  source 1..452: note = Synthetic Sequence
FRGPLLPNRPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 57 - protein, 452 aa, synthetic construct
  source 1..452: note = Synthetic Sequence
FRGPLLPNRPFTTVWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 58 - protein, 433 aa, synthetic construct
  source 1..433: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 59 - protein, 431 aa, synthetic construct
  source 1..431: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 60 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 61 - protein, 451 aa, synthetic construct
  source 1..451: note = Synthetic Sequence
APPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 62 - protein, 449 aa, synthetic construct
  source 1..449: note = Synthetic Sequence
PVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 63 - protein, 432 aa, synthetic construct
  source 1..432: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 64 - protein, 429 aa, synthetic construct
  source 1..429: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVC

SEQ ID NO: 65 - protein, 426 aa, synthetic construct
  source 1..426: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIAD

SEQ ID NO: 66 - protein, 423 aa, synthetic construct
  source 1..423: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVC

SEQ ID NO: 67 - protein, 420 aa, synthetic construct
  source 1..420: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAV

SEQ ID NO: 68 - protein, 433 aa, synthetic construct
  source 1..433: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 69 - protein, 435 aa, synthetic construct
  source 1..435: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPP

SEQ ID NO: 70 - protein, 436 aa, synthetic construct
  source 1..436: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPM

SEQ ID NO: 71 - protein, 437 aa, synthetic construct
  source 1..437: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPME

SEQ ID NO: 72 - protein, 438 aa, synthetic construct
  source 1..438: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMET

SEQ ID NO: 73 - protein, 439 aa, synthetic construct
  source 1..439: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETE

SEQ ID NO: 74 - protein, 454 aa, synthetic construct
  source 1..454: note = Synthetic Sequence
NFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 75 - protein, 452 aa, synthetic construct
  source 1..452: note = Synthetic Sequence
RAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 76 - protein, 450 aa, synthetic construct
  source 1..450: note = Synthetic Sequence
PPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 77 - protein, 428 aa, synthetic construct
  source 1..428: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 78 - protein, 429 aa, synthetic construct
  source 1..429: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCID

SEQ ID NO: 79 - protein, 430 aa, synthetic construct
  source 1..430: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 80 - protein, 431 aa, synthetic construct
  source 1..431: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 81 - protein, 441 aa, synthetic construct
  source 1..441: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 82 - protein, 443 aa, synthetic construct
  source 1..443: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 83 - protein, 445 aa, synthetic construct
  source 1..445: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 84 - protein, 447 aa, synthetic construct
  source 1..447: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNA

SEQ ID NO: 85 - protein, 449 aa, synthetic construct
  source 1..449: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASP

SEQ ID NO: 86 - protein, 451 aa, synthetic construct
  source 1..451: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPST

SEQ ID NO: 87 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGGWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 88 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGAWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 89 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGCWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 90 - protein, 455 aa, synthetic construct
  source 1..455: note = Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGDWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 91 - protein, 431 aa, synthetic construct
  source 1..431: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSNTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 92 - protein, 431 aa, synthetic construct
  source 1..431: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 93 - protein, 431 aa, synthetic construct
  source 1..431: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGAWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 94 - protein, 431 aa, synthetic construct
  source 1..431: note = Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGGWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 95 - protein, 431 aa, synthetic construct
  source 1..431: note = Synthetic Sequence
FRGPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 96 - protein, 431 aa, synthetic construct; identical to SEQ ID NO: 95
  source 1..431: note = Synthetic Sequence